(19) United States
(12) Patent Application Publication
(10) Pub. No.: US 2024/0065527 A1
(43) Pub. Date: Feb. 29, 2024

(54) MEDICAL SUPPORT DEVICE, ENDOSCOPE, MEDICAL SUPPORT METHOD, AND PROGRAM

(71) Applicant: FUJIFILM Corporation, Tokyo (JP)

(72) Inventor: Kentaro OSHIRO, Kanagawa (JP)

(73) Assignee: FUJIFILM Corporation, Tokyo (JP)

(21) Appl. No.: 18/447,293

(22) Filed: Aug. 9, 2023

(30) Foreign Application Priority Data
Aug. 30, 2022 (JP) 2022-137264

(51) Int. Cl. A61B 1/00

(52) U.S. Cl. CPC A61B 1/0005

(57) ABSTRACT

A medical support device includes a processor. The processor acquires endoscope-related information that is related to an endoscope and displays, on a display device, at least one image selected according to the endoscope-related information among a plurality of images in which an observation target observed through the endoscope is divided into a plurality of regions and which are represented in different aspects.

### CROSS-REFERENCE TO RELATED APPLICATIONS

This application claims priority under 35 USC 119 from Japanese Patent Application No. 2022-137264 filed on Aug. 30, 2022, the disclosure of which is incorporated by reference herein.

### BACKGROUND

### 1. Technical Field

A technology of the present disclosure relates to a medical support device, an endoscope, a medical support method, and a program.

### 2. Related Art

WO2020/054543A discloses a medical image processing device comprising an image acquisition unit that acquires a plurality of time-series images including a subject image, a suitability determination unit that determines whether or not the image obtained from the image acquisition unit is an image unsuitable for recognition, a movement estimation unit that estimates a movement from two or more images obtained from the image acquisition unit, an action determination unit that determines the action of a user on the basis of movement information obtained from the movement estimation unit, a classification unit that recognizes the image obtained from the image acquisition unit and performs a classification process, and a notification control unit that controls notification information on the basis of action information obtained from the action determination unit and a classification result obtained from the classification unit.

JP2004-350793A discloses a medical image recording device which is connected to an endoscope system outputting a captured endoscopic image and comprises a message generation unit that generates a message and a combination unit that combines an endoscopic image input from an imaging device with the message generated by the message generation unit.

JP2012-239815A discloses an endoscope system comprising a screening image acquisition unit that acquires a screening image used during screening for detecting a potential lesion part on a subject, a detailed diagnostic image acquisition unit that acquires a detailed diagnostic image which is different from the screening image and is used to identify whether or not the potential lesion part is a lesion portion, an observation distance calculation unit that calculates an observation distance indicating a distance from an observation region on the subject, and a display control unit that displays the screening image on a display unit in a case in which the observation distance is equal to or greater than a predetermined value and displays the detailed diagnostic image on the display unit in a case in which the observation distance is less than the predetermined value.

WO2019/244255A discloses an endoscopic image processing device comprising an image acquisition unit that acquires an image of a subject captured by an endoscope, a display output unit that outputs a display image including at least the image acquired by the image acquisition unit to a display unit, a region-of-interest detection unit that detects a region of interest included in the image acquired by the image acquisition unit, a detection interruption determination unit that determines whether or not the detection of the region of interest by the region-of-interest detection unit has been interrupted, and a display determination unit that performs display propriety determination which is determination of whether or not to display, on the display unit, support information for performing support such that the region of interest whose detection has been interrupted is returned to a screen of the display unit in a case in which interruption determination which is a determination result of the detection interruption determination unit indicating that the detection of the region of interest has been interrupted is obtained. In a case in which it is determined that the support information is displayed in the display propriety determination, the display output unit outputs an image that further includes the support information as the display image to the display unit. In a case in which it is determined that the support information is not displayed in the display propriety determination, the display output unit outputs an image that does not include the support information as the display image to the display unit.

WO2018/221033A discloses a medical image processing device including an image acquisition unit that acquires a medical image including a subject, a display unit that displays the medical image in a first display region, and a display control unit that performs control to display notification information to be notified to a user on the display unit or control not to display the notification information on the display unit. The display control unit performs control to display the notification information in a second display region different from the first display region or control to remove the notification information that is being displayed.

### SUMMARY

An embodiment according to the technology of the present disclosure is to provide a medical support device, an endoscope, a medical support method, and a program that enable a user to easily understand a plurality of parts in an observation target observed through the endoscope.

According to a first aspect of the technology of the present disclosure, there is provided a medical support device comprising a processor. The processor acquires endoscope-related information that is related to an endoscope and displays, on a display device, at least one image selected according to the endoscope-related information among a plurality of images in which an observation target observed through the endoscope is divided into a plurality of regions and which are represented in different aspects.

According to a second aspect of the technology of the present disclosure, in the medical support device according to the first aspect, the plurality of images may have different amounts of visual information.

According to a third aspect of the technology of the present disclosure, in the medical support device according to the second aspect, the amount of information may be classified into a first amount of information and a second amount of information that is less than the first amount of information, the endoscope-related information may include difficulty information that is capable of specifying a difficulty of a technique using the endoscope and/or a difficulty of mental rotation, and the processor may switch between the image with the first amount of information and the image with the second amount of information as the image to be displayed on the display device according to the difficulty information.

According to a fourth aspect of the technology of the present disclosure, in the medical support device according to any one of the first to third aspects, the plurality of images may be classified into a simple image in a simple format and a detailed image in a format that is more detailed than the simple image.

According to a fifth aspect of the technology of the present disclosure, in the medical support device according to the fourth aspect, the endoscope-related information may include difficulty information that is capable of specifying a difficulty of a technique using the endoscope and/or a difficulty of mental rotation, and the processor may switch between the simple image and the detailed image as the image to be displayed on the display device according to the difficulty information.

According to a sixth aspect of the technology of the present disclosure, in the medical support device according to any one of the first to fifth aspects, the observation target may be a luminal organ, the plurality of images may be a plurality of schematic views including a first schematic view, a second schematic view, and a third schematic view, the first schematic view may be a view showing a schematic aspect of at least one route for observing the luminal organ, the second schematic view may be a perspective view showing a schematic aspect of the luminal organ, and the third schematic view may be a view showing an aspect in which the luminal organ is schematically developed.

According to a seventh aspect of the technology of the present disclosure, in the medical support device according to the sixth aspect, the plurality of regions may be classified into a major category and a minor category included in the major category. In at least one of the first schematic view, the second schematic view, or the third schematic view, the major category, the minor category, or both the major category and the minor category may be represented.

According to an eighth aspect of the technology of the present disclosure, in the medical support device according to any one of the first to seventh aspects, the endoscope-related information may include information that is capable of specifying content of an operation corresponding to the endoscope.

According to a ninth aspect of the technology of the present disclosure, in the medical support device according to any one of the first to eighth aspects, the endoscope-related information may include information that is capable of specifying an operator of the endoscope.

According to a tenth aspect of the technology of the present disclosure, in the medical support device according to any one of the first to ninth aspects, the endoscope may generate an endoscopic image including the observation target, and the endoscope-related information may be information generated on the basis of the endoscopic image.

According to an eleventh aspect of the technology of the present disclosure, in the medical support device according to any one of the first to tenth aspects, the endoscope may generate an endoscopic image including the observation target, the processor may classify the plurality of regions into an observed region which has been observed through the endoscope and an unobserved region which has not been observed through the endoscope on the basis of the endoscopic image, and the observed region and the unobserved region may be displayed to be distinguishable from each other in the at least one image.

According to a twelfth aspect of the technology of the present disclosure, in the medical support device according to the eleventh aspect, the observation target may be a luminal organ, and the plurality of images may include a first image in which a position of the endoscope in the luminal organ and the plurality of regions are comparable from each other and a second image in which the observed region and the unobserved region in the luminal organ are distinguishable from each other.

According to a thirteenth aspect of the technology of the present disclosure, in the medical support device according to the eleventh aspect, the observation target may be a luminal organ, and the plurality of images may include a third image in which the observed region and the unobserved region in the luminal organ are distinguishable from each other and at least one fourth image in which the observed region and the unobserved region in the luminal organ are distinguishable from each other in more detail than the third image.

According to a fourteenth aspect of the technology of the present disclosure, in the medical support device according to the thirteenth aspect, the plurality of images may include, as the fourth image, a fourth schematic view showing a schematic aspect of at least one route for observing the luminal organ and a fifth schematic view showing an aspect in which the luminal organ is schematically developed.

According to a fifteenth aspect of the technology of the present disclosure, in the medical support device according to the thirteenth aspect or the fourteenth aspect, the third image and the at least one fourth image may be selectively displayed on the display device, using the third image as a starting point.

According to a sixteenth aspect of the technology of the present disclosure, in the medical support device according to the first aspect, the processor may output unobserved information capable of specifying that an unobserved region, which has not been observed through the endoscope, is present in the plurality of regions along a first route determined from an upstream side to a downstream side in an insertion direction of the endoscope inserted into a body in a case in which a first part on the upstream side and a second part on the downstream side in the insertion direction are sequentially recognized and may output the unobserved information along a second route determined from the downstream side to the upstream side in the insertion direction in a case in which a third part on the downstream side and a fourth part on the upstream side in the insertion direction are sequentially recognized.

According to a seventeenth aspect of the technology of the present disclosure, there is provided an endoscope comprising: the medical support device according to any one of the first to sixteenth aspects; and an image acquisition device that acquires an endoscopic image including the observation target.

According to an eighteenth aspect of the technology of the present disclosure, there is provided a medical support method comprising: acquiring endoscope-related information that is related to an endoscope; and displaying, on a display device, at least one image selected according to the endoscope-related information among a plurality of images in which an observation target observed through the endoscope is divided into a plurality of regions and which are represented in different aspects.

According to a nineteenth aspect of the technology of the present disclosure, there is provided a program that causes a computer to execute a process comprising: acquiring endoscope-related information that is related to an endoscope; and displaying, on a display device, at least one image selected according to the endoscope-related information among a plurality of images in which an observation target observed through the endoscope is divided into a plurality of regions and which are represented in different aspects.

### BRIEF DESCRIPTION OF THE DRAWINGS

Exemplary embodiments of the technology of the disclosure will be described in detail based on the following figures, wherein:

FIG. 1 is a conceptual diagram illustrating an example of an aspect in which an endoscope system is used;

FIG. 2 is a conceptual diagram illustrating an example of an overall configuration of the endoscope system;

FIG. 3 is a block diagram illustrating an example of a hardware configuration of an electrical system of the endoscope system;

FIG. 4 is a block diagram illustrating an example of functions of main units of a processor included in an endoscope;

FIG. 5 is a conceptual diagram illustrating an example of a correlation among an endoscope, an image acquisition unit, and an endoscope recognition unit.

FIG. 6 is a conceptual diagram illustrating an example of a correlation among the endoscope recognition unit, a control unit, and a display device;

FIG. 7 is a conceptual diagram illustrating an example of a correlation among the endoscope, the image acquisition unit, a part recognition unit, and an NVM;

FIG. 8 is a conceptual diagram illustrating an example of a configuration of a recognition part check table;

FIG. 9 is a conceptual diagram illustrating an example of a configuration of an importance table;

FIG. 10 is a conceptual diagram illustrating an example of a first medical support image displayed on a screen of the display device;

FIG. 11 is a conceptual diagram illustrating an example of a second medical support image displayed on the screen of the display device;

FIG. 12 is a conceptual diagram illustrating an example of a third medical support image displayed on the screen of the display device;

FIG. 13A is a flowchart illustrating an example of a flow of a medical support process.

FIG. 13B is a flowchart illustrating an example of the flow of the medical support process;

FIG. 14 is a conceptual diagram illustrating an example of an aspect in which a reference image, the first medical support image, the second medical support image, and the third medical support image are selectively displayed on a screen, using the reference image as a starting point; and

FIG. 15 is a conceptual diagram illustrating an example of an aspect in which the third medical support image and the reference image are displayed side by side on the screen.

### DETAILED DESCRIPTION

Hereinafter, examples of embodiments of a medical support device, an endoscope, a medical support method, and a program according to the technology of the present disclosure will be described with reference to the accompanying drawings.

First, terms used in the following description will be described.

CPU is an abbreviation of “central processing unit”. GPU is an abbreviation of “graphics processing unit”. RAM is an abbreviation of “random access memory”. NVM is an abbreviation of “non-volatile memory”. EEPROM is an abbreviation of “electrically erasable programmable read-only memory”. ASIC is an abbreviation of “application specific integrated circuit”. PLD is an abbreviation of “programmable logic device”. FPGA is an abbreviation of “field-programmable gate array”. SoC is an abbreviation of “system-on-a-chip”. SSD is an abbreviation of “solid state drive”. USB is an abbreviation of “universal serial bus”. HDD is an abbreviation of “hard disk drive”. EL is an abbreviation of “electro-luminescence”. CMOS is an abbreviation of “complementary metal oxide semiconductor”. CCD is an abbreviation of “charge coupled device”. AI is an abbreviation of “artificial intelligence”. BLI is an abbreviation of “blue light imaging”. LCI is an abbreviation of “linked color imaging”. I/F is an abbreviation of “interface”. FIFO is an abbreviation of “first in first out”. The ID refers to an abbreviation of “identification”.

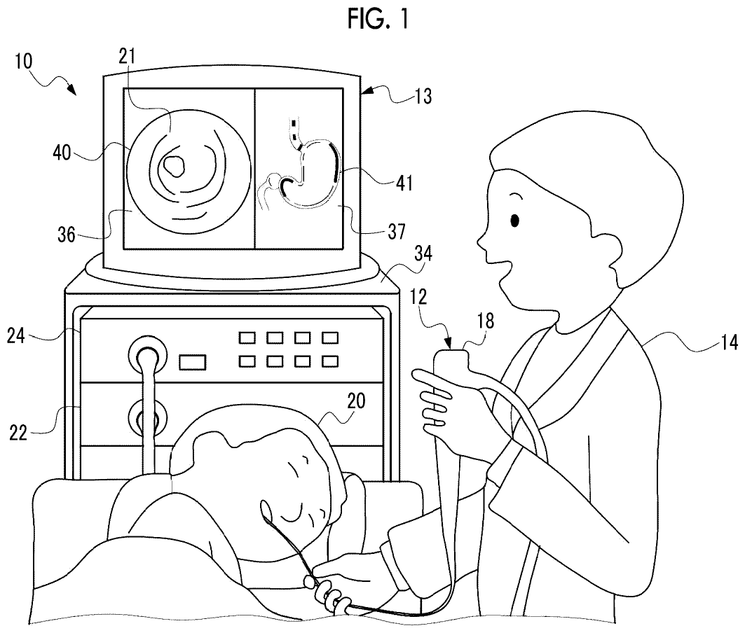

For example, as illustrated in FIG. 1, an endoscope system 10 comprises an endoscope 12 and a display device 13. The endoscope 12 is used by a doctor 14 in endoscopy. The endoscope 12 is connected to a communication device (not illustrated) such that it can communicate, and information obtained by the endoscope 12 is transmitted to the communication device. The communication device receives the information transmitted from the endoscope 12 and performs a process using the received information (for example, a process of recording the information on an electronic medical record or the like).

The endoscope 12 comprises an endoscope main body 18. The endoscope 12 is a device for performing a medical treatment on an observation target 21 (for example, an upper digestive organ) included in a body of a subject 20 (for example, a patient) using the endoscope main body 18. The observation target 21 is an object observed by the doctor 14. The endoscope main body 18 is inserted into the body of the subject 20. The endoscope 12 directs the endoscope main body 18 inserted into the body of the subject 20 to image the observation target 21 in the body of the subject 20 and performs various medical treatments on the observation target 21 as necessary. The endoscope 12 is an example of an “endoscope” according to the technology of the present disclosure.

The endoscope 12 images the inside of the body of the subject 20 to acquire an image showing an aspect of the inside of the body and outputs the image. In the example illustrated in FIG. 1, an upper endoscope is given as an example of the endoscope 12. In addition, the upper endoscope is only an example, and the technology of the present disclosure can be established even in a case in which the endoscope 12 is another type of endoscope such as a lower gastrointestinal endoscope or a bronchoscope.

Further, in this embodiment, the endoscope 12 is an endoscope having an optical imaging function that irradiates the inside of the body with light and captures light reflected by the observation target 21. However, this is only an example, and the technology of the present disclosure is established even in a case in which the endoscope 12 is an ultrasonic endoscope.

The endoscope 12 comprises a control device 22 and a light source device 24. The control device 22 and the light source device 24 are installed in a wagon 34. A plurality of tables are provided in the wagon 34 along the vertical direction, and the control device 22 and the light source device 24 are installed from a lower table to an upper table. In addition, a display device 13 is installed on the uppermost table in the wagon 34.

The display device 13 displays various types of information including images. An example of the display device 13 is a liquid crystal display or an EL display. In addition, a tablet terminal with a display may be used instead of the display device 13 or together with the display device 13.

A plurality of screens are displayed side by side on the display device 13. In the example illustrated in FIG. 1, screens 36 and 37 are illustrated. An endoscopic image 40 obtained by the endoscope 12 is displayed on the screen 36. The endoscopic image 40 is an example of an “endoscopic image” according to the technology of the present disclosure.

The observation target 21 is included in the endoscopic image 40. The endoscopic image 40 is an image generated by imaging the observation target 21 with the endoscope 12 in the body of the subject 20. An example of the observation target 21 is the upper digestive organ. Hereinafter, for convenience of explanation, the stomach will be described as an example of the upper digestive organ. The stomach is an example of a “luminal organ” according to the technology of the present disclosure. In addition, the stomach is only an example, and the observation target 21 may be any region that can be imaged by the endoscope 12. A luminal organ, such as a large intestine, a small intestine, a duodenum, an esophagus, or a bronchus, is given as an example of the region that can be imaged by the endoscope 12.

A moving image including the endoscopic images 40 of a plurality of frames is displayed on the screen 36. That is, the endoscopic images 40 of a plurality of frames are displayed on the screen 36 at a predetermined frame rate (for example, several tens of frames/sec).

A medical support image 41 is displayed on the screen 37. The medical support image 41 is an image that is referred to by the doctor 14 during endoscopy. The medical support image 41 is referred to by the doctor 14 to check a plurality of parts that are scheduled to be observed during the endoscopy. In addition, the medical support image 41 includes information indicating whether or not the omission of observation has occurred in a plurality of parts scheduled to be observed during the endoscopy, and the doctor 14 ascertains whether or not the omission of the observation has occurred in the plurality of parts with reference to the medical support image 41.

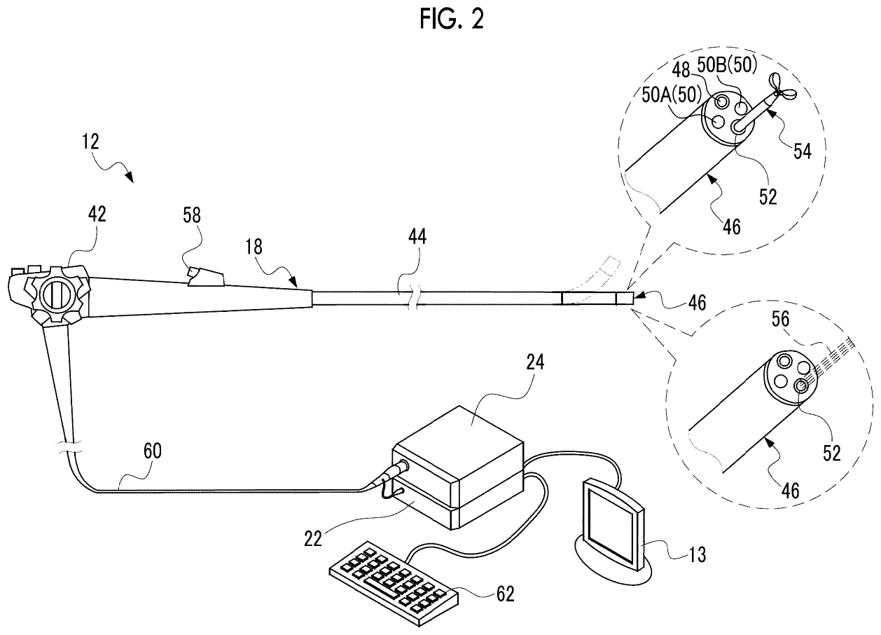

For example, as illustrated in FIG. 2, the endoscope 12 comprises an operation portion 42 and an insertion portion 44. The insertion portion 44 is partially curved by the operation of the operation portion 42. The insertion portion 44 is inserted while being curved according to the shape of the observation target 21 (for example, the shape of the stomach) in response to the operation of the operation portion 42 by the doctor 14.

A distal end part 46 of the insertion portion 44 is provided with a camera 48, an illumination device 50, and a treatment opening 52. The camera 48 is a device that images the inside of the body of the subject 20 to acquire the endoscopic image 40 as a medical image. The camera 48 is an example of an “image acquisition device” according to the technology of the present disclosure. An example of the camera 48 is a CMOS camera. However, this is only an example, and the camera 48 may be other types of cameras such as CCD cameras.

The illumination device 50 has illumination windows 50A and 50B. The illumination device 50 emits light through the illumination windows 50A and 50B. Examples of the type of light emitted from the illumination device 50 include visible light (for example, white light) and invisible light (for example, near-infrared light). In addition, the illumination device 50 emits special light through the illumination windows 50A and 50B. Examples of the special light include light for BLI and/or light for LCI. The camera 48 images the inside of the body of the subject 20 using an optical method in a state in which the illumination device 50 irradiates the inside of the body of the subject 20 with light.

The treatment opening 52 is used as a treatment tool protruding port through which a treatment tool 54 protrudes from the distal end part 46, a suction port for sucking, for example, blood and internal filth, and a delivery port for sending out a fluid 56.

The treatment tool 54 protrudes from the treatment opening 52 in response to the operation of the doctor 14. The treatment tool 54 is inserted into the insertion portion 44 through a treatment tool insertion opening 58. The treatment tool 54 passes through the insertion portion 44 through the treatment tool insertion opening 58 and protrudes from the treatment opening 52 into the body of the subject 20. In the example illustrated in FIG. 2, as the treatment tool 54, forceps protrude from the treatment opening 52. The forceps are only an example of the treatment tool 54, and other examples of the treatment tool 54 include a wire, a scalpel, and an ultrasound probe.

A suction pump (not illustrated) is connected to the endoscope main body 18, and blood, internal filth, and the like of the observation target 21 are sucked by the suction force of the suction pump through the treatment opening 52. The suction force of the suction pump is controlled in response to an instruction given from the doctor 14 to the endoscope 12 through, for example, the operation portion 42.

A supply pump (not illustrated) is connected to the endoscope main body 18, and the fluid 56 (for example, gas and/or liquid) is supplied into the endoscope main body 18 by the supply pump. The fluid 56 supplied from the supply pump to the endoscope main body 18 is sent out through the treatment opening 52. Gas (for example, air) and liquid (for example, physiological saline) are selectively sent out as the fluid 56 from the treatment opening 52 into the body in response to an instruction given from the doctor 14 to the endoscope 12 through the operation portion 42 or the like. The amount of the fluid 56 sent out is controlled in response an instruction given from the doctor 14 to the endoscope 12 through the operation portion 42 or the like.

In addition, here, an example of the form in which the treatment opening 52 is used as the treatment tool protruding port, the suction port, and the delivery port is given. However, this is only an example, and the treatment tool protruding port, the suction port, and the delivery port may be separately provided in the distal end part 46, or the treatment tool protruding port and an opening that serves as the suction port and the delivery port may be provided in the distal end part 46.

The endoscope main body 18 is connected to the control device 22 and the light source device 24 through a universal cord 60. The display device 13 and a receiving device 62 are connected to the control device 22. The receiving device 62 receives an instruction from the user and outputs the received instruction as an electric signal. In the example illustrated in FIG. 2, a keyboard is given as an example of the receiving device 62. However, this is only an example, and the receiving device 62 may be, for example, a mouse, a touch panel, a foot switch and/or a microphone.

The control device 22 controls the entire endoscope 12. For example, the control device 22 controls the light source device 24, transmits and receives various signals to and from the camera 48, or displays various types of information on the display device 13. The light source device 24 emits light under the control of the control device 22 and supplies the light to the illumination device 50. A light guide is provided in the illumination device 50, and the light supplied from the light source device 24 is emitted from the illumination windows 50A and 50B through the light guide. The control device 22 directs the camera 48 to perform imaging, acquires the endoscopic image 40 (see FIG. 1) from the camera 48, and outputs the endoscopic image 40 to a predetermined output destination (for example, the display device 13).

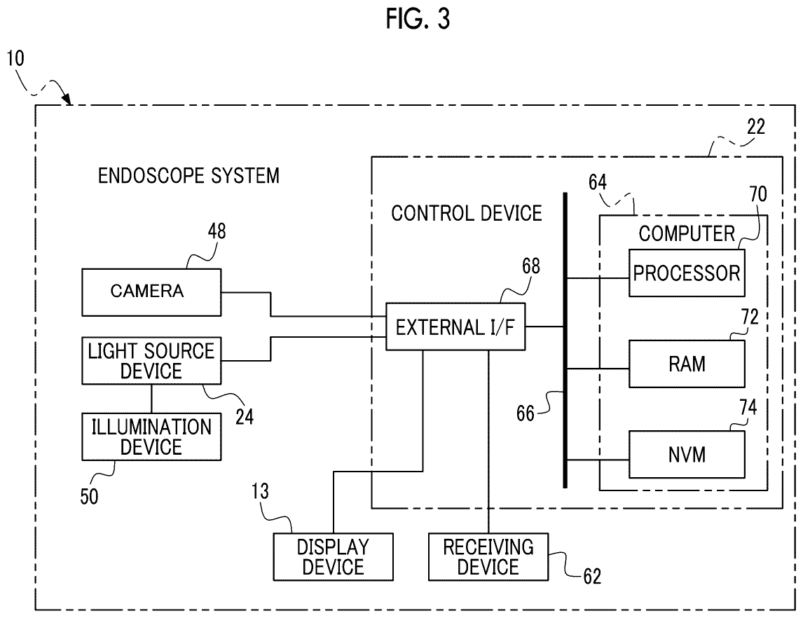

For example, as illustrated in FIG. 3, the control device 22 comprises a computer 64. The computer 64 is an example of a “medical support device” and a “computer” according to the technology of the present disclosure. The computer 64 comprises a processor 70, a RAM 72, and an NVM 74, and the processor 70, the RAM 72, and the NVM 74 are electrically connected to each other. The processor 70 is an example of a “processor” according to the technology of the present disclosure.

The control device 22 comprises the computer 64, a bus 66, and an external OF 68. The computer 64 comprises the processor 70, the RAM 72, and the NVM 74. The processor 70, the RAM 72, the NVM 74, and the external OF 68 are connected to the bus 66.

For example, the processor 70 includes a CPU and a GPU and controls the entire control device 22. The GPU operates under the control of the CPU and is in charge of, for example, performing various processes of a graphic system and performing calculation using a neural network. In addition, the processor 70 may be one or more CPUs with which the functions of the GPU have been integrated or may be one or more CPUs with which the functions of the GPU have not been integrated.

The RAM 72 is a memory that temporarily stores information and is used as a work memory by the processor 70. The NVM 74 is a non-volatile storage device that stores, for example, various programs and various parameters. An example of the NVM 74 is a flash memory (for example, an EEPROM and/or an SSD). In addition, the flash memory is only an example and may be other non-volatile storage devices, such as HDD s, or a combination of two or more types of non-volatile storage devices.

The external I/F 68 transmits and receives various types of information between a device (hereinafter, also referred to as an “external device”) outside the control device 22 and the processor 70. An example of the external I/F 68 is a USB interface.

As one of the external devices, the camera 48 is connected to the external I/F 68, and the external I/F 68 transmits and receives various types of information between the camera 48 and the processor 70. The processor 70 controls the camera 48 through the external I/F 68. In addition, the processor 70 acquires the endoscopic image 40 (see FIG. 1) obtained by imaging the inside of the subject 20 with the camera 48 through the external I/F 68.

As one of the external devices, the light source device 24 is connected to the external I/F 68, and the external I/F 68 transmits and receives various types of information between the light source device 24 and the processor 70. The light source device 24 supplies light to the illumination device 50 under the control of the processor 70. The illumination device 50 performs irradiation with the light supplied from the light source device 24.

As one of the external devices, the display device 13 is connected to the external I/F 68, and the processor 70 controls the display device 13 through the external I/F 68 such that the display device 13 displays various types of information.

As one of the external devices, the receiving device 62 is connected to the external I/F 68. The processor 70 acquires the instruction received by the receiving device 62 through the external I/F 68 and performs a process corresponding to the acquired instruction.

However, in general, in endoscopy, a lesion is detected by using an image recognition process (for example, an AI-type image recognition process). In some cases, for example, a treatment for cutting out the lesion is performed. In addition, in endoscopy, since the doctor 14 performs the operation of the insertion portion 44 of the endoscope 12 and the differentiation of a lesion at the same time, the burden on the doctor 14 is large, and there is a concern that the lesion will be overlooked. In order to prevent the lesion from being overlooked, it is important that a plurality of parts scheduled in advance in the observation target 21 are recognized by the image recognition process without omission.

A method for display the medical support image on the display device 13 is considered as a method for allowing the doctor 14 to check whether or not a plurality of parts scheduled in advance in the observation target 21 have been recognized by the image recognition process without omission. The medical support image is an image that is used for the doctor 14 to understand which part has been recognized by the image recognition process and is referred to by the doctor 14 during endoscopy. However, it is expected that the doctor 14 will not be able to fully understand the content of the medical support image displayed on the display device 13, depending on the difficulty of the technique using the endoscope 12 and/or the difficulty of the mental rotation by the doctor 14. However, in a case in which the medical support image is not displayed on the display device 13 at all, it is difficult for the doctor 14 to check whether or not a plurality of parts scheduled in advance in the observation target 21 have been recognized by the image recognition process without omission.

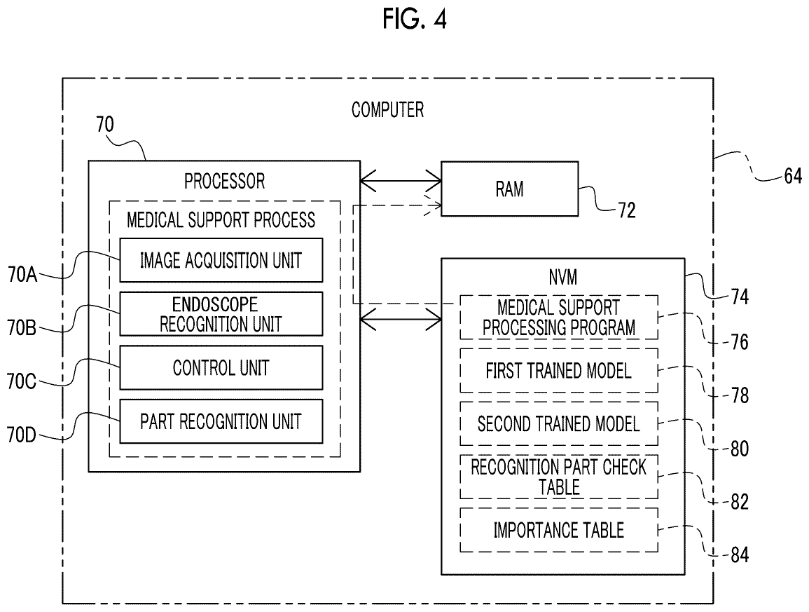

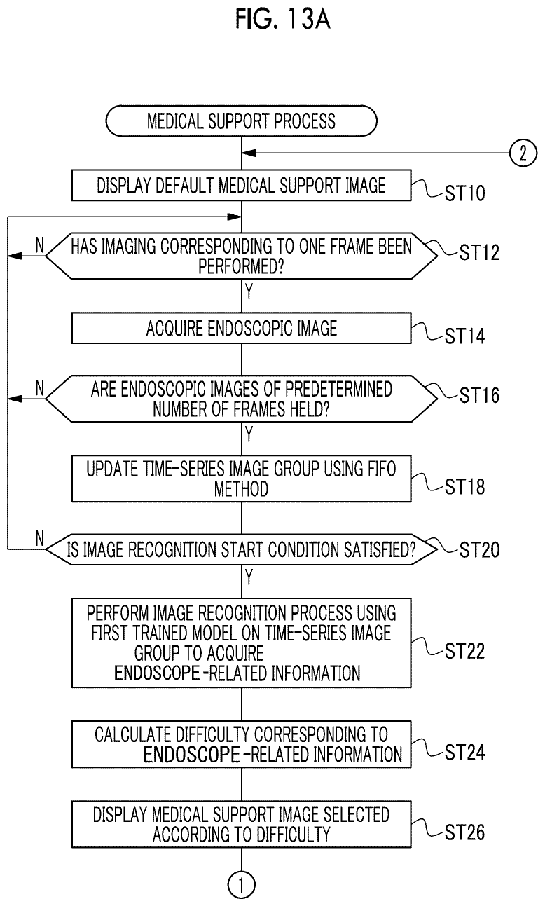

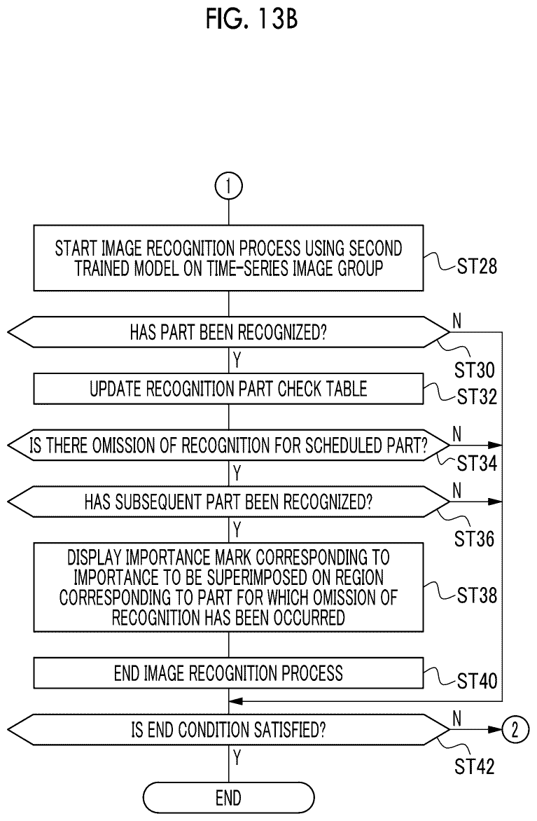

Therefore, in view of this, in this embodiment, the medical support process is performed by the processor 70 of the control device 22 in order to suppress the omission of the recognition of a plurality of parts scheduled in advance by the image recognition process regardless of the difficulty of the technique using the endoscope 12 and/or the difficulty of the mental rotation by the doctor 14 (see FIGS. 4, 13A, and 13B). Further, in this embodiment, the omission of the recognition is synonymous with the above-described omission of observation.

The medical support process includes a process including the acquisition of endoscope-related information related to the endoscope 12 and the display of at least one image selected from a plurality of images in which the observation target 21 observed through the endoscope 12 is divided into a plurality of regions and which are represented in different aspects on the display device. Hereinafter, the medical support process will be described in more detail.

For example, as illustrated in FIG. 4, a medical support processing program 76 is stored in the NVM 74. The medical support processing program 76 is an example of a “program” according to the technology of the present disclosure. The processor 70 reads the medical support processing program 76 from the NVM 74 and executes the read medical support processing program 76 on the RAM 72. The processor 70 operates as an image acquisition unit 70A, an endoscope recognition unit 70B, a control unit 70C, and a part recognition unit 70D according to the medical support processing program 76 executed on the RAM 72 to achieve the medical support process.

A first trained model 78 and a second trained model 80 are stored in the NVM 74. In this embodiment, the endoscope recognition unit 70B and the part recognition unit 70D perform an AI-type image recognition process as an image recognition process for object detection. The AI-type image recognition process performed by the endoscope recognition unit 70B indicates an image recognition process using the first trained model 78. In addition, the AI-type image recognition process performed by the part recognition unit 70D indicates an image recognition process using the second trained model 80. Hereinafter, for convenience of explanation, in a case in which the first trained model 78 and the second trained model 80 do not need to be distinguished from each other for description, they are also referred to as “trained models” without reference numerals.

The trained model is a mathematical model for object detection and is obtained by performing machine learning on the neural network in advance to optimize the neural network. Hereinafter, the image recognition process using the trained model will be described as a process that is actively performed by the trained model. That is, in the following description, for convenience of explanation, the trained model is considered as a function of performing a process on input information and outputting the result of the process.

A recognition part check table 82 and an importance table 84 are stored in the NVM 74. Both the recognition part check table 82 and the importance table 84 are used by the control unit 70C.

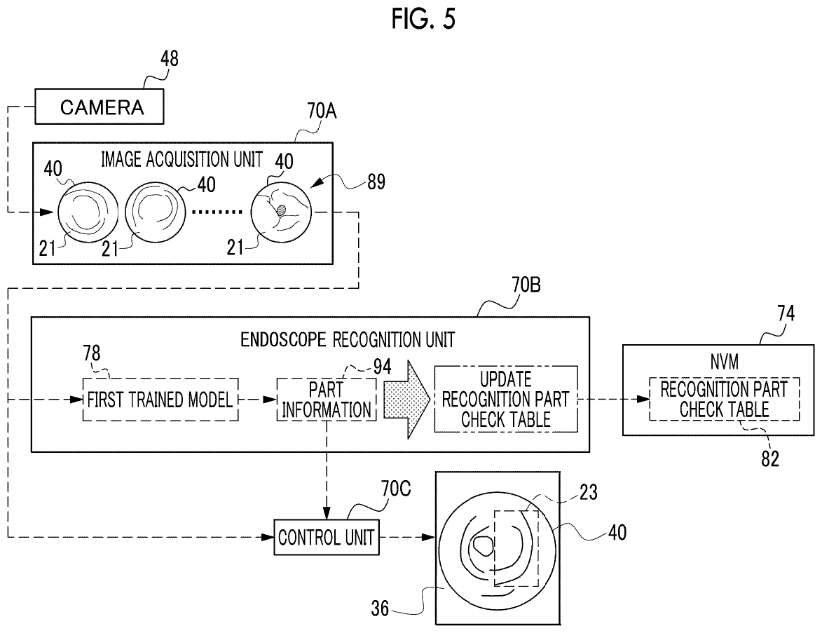

For example, as illustrated in FIG. 5, the image acquisition unit 70A acquires the endoscopic image 40, which has been captured by the camera 48 at an imaging frame rate (for example, several tens of frames/sec), from the camera 48 frame by frame.

The image acquisition unit 70A holds a time-series image group 89. The time-series image group 89 is a plurality of time-series endoscopic images 40 including the observation target 21. The time-series image group 89 includes, for example, the endoscopic images 40 of a predetermined number of frames (for example, a predetermined number of frames within a range of several tens to several hundreds of frames). The image acquisition unit 70A updates the time-series image group 89 using a FIFO method whenever the endoscopic image 40 is acquired from the camera 48.

Here, an example of the form in which the time-series image group 89 is held and updated by the image acquisition unit 70A has been described. However, this is only an example. For example, the time-series image group 89 may be held in a memory, such as the RAM 72, that is connected to the processor 70 and then updated.

The endoscope recognition unit 70B performs the image recognition process using the first trained model 78 on the time-series image group 89 to detect, for example, the state of the endoscope 12. The first trained model 78 is optimized by performing machine learning on the neural network using first training data. An example of the first training data is training data in which a plurality of images obtained in time series by imaging the inside of the body with the camera 48 are example data and endoscope-related information 90 related to the endoscope 12 is correct answer data. In addition, here, an example of the form in which only one first trained model 78 is used by the endoscope recognition unit 70B has been described. However, this is only an example. For example, the first trained model 78 selected from a plurality of first trained models 78 may be used by the endoscope recognition unit 70B. In this case, each first trained model 78 may be created by performing machine learning specialized for each type of endoscopy, and the first trained model 78 corresponding to the type of endoscopy which is currently being performed (here, for example, the type of the endoscope 12) may be selected and used by the endoscope recognition unit 70B.

The endoscope recognition unit 70B acquires the time-series image group 89 and generates the endoscope-related information 90 on the basis of the acquired time-series image group 89. In order to generate the endoscope-related information 90, the endoscope recognition unit 70B inputs the time-series image group 89 to the first trained model 78. Then, the first trained model 78 outputs the endoscope-related information 90 corresponding to the input time-series image group 89. The endoscope recognition unit 70B acquires the endoscope-related information 90 output from the first trained model 78. The endoscope-related information 90 acquired by the endoscope recognition unit 70B is information related to the endoscope 12 that is currently being used. The endoscope-related information 90 is an example of “endoscope-related information” according to the technology of the present disclosure.

The endoscope-related information 90 is information that can specify the content of the operation for the endoscope 12 and that can specify the difficulty of the technique using the endoscope 12 and/or the difficulty of the mental rotation and includes treatment tool information 90A, operation speed information 90B, positional information 90C, shape information 90D, fluid delivery information 90E, and the like. The treatment tool information 90A, the operation speed information 90B, the positional information 90C, the shape information 90D, the fluid delivery information 90E, and the like are also information that can specify the content of the operation for the endoscope 12. The treatment tool information 90A, the operation speed information 90B, the positional information 90C, the shape information 90D, the fluid delivery information 90E, and the like are examples of “difficulty information” according to the technology of the present disclosure.

The treatment tool information 90A is information related to the treatment tool 54 (see FIG. 2). Examples of the information related to the treatment tool 54 include information indicating whether or not the treatment tool 54 is being used and information indicating the type of the treatment tool 54 that is being used. The operation speed information 90B is information related to the operation speed of the distal end part 46 (see FIG. 2) of the endoscope 12 (for example, information related to the speed represented in units of “millimeters/second”).

The positional information 90C is information related to the position of the distal end part 46 of the endoscope 12. An example of the information related to the position of the distal end part 46 of the endoscope 12 is three-dimensional coordinates indicating a position within the observation target 21 in a case in which a reference position (for example, a portion of the entrance of the stomach) is the origin. The shape information 90D is information related to the shape of the insertion portion 44 of the endoscope 12. Examples of the information related to the shape of the insertion portion 44 of the endoscope 12 include information indicating a direction in which the insertion portion 44 is curved and/or the degree of curvature of the insertion portion 44.

The fluid delivery information 90E is information related to the delivery of the fluid 56 (see FIG. 2). The information related to the delivery of the fluid 56 indicates, for example, information related to the delivery amount of the fluid 56 per unit time (for example, information related to the delivery amount represented in units of “milliliters/sec”). The fluid delivery information 90E includes air supply amount information 90E1 and water supply amount information 90E2. The air supply amount information 90E1 is information related to the supply amount of gas (for example, information related to the supply amount of gas per unit time). The water supply amount information 90E2 is information related to the supply amount of liquid (for example, information related to the supply amount of liquid per unit time).

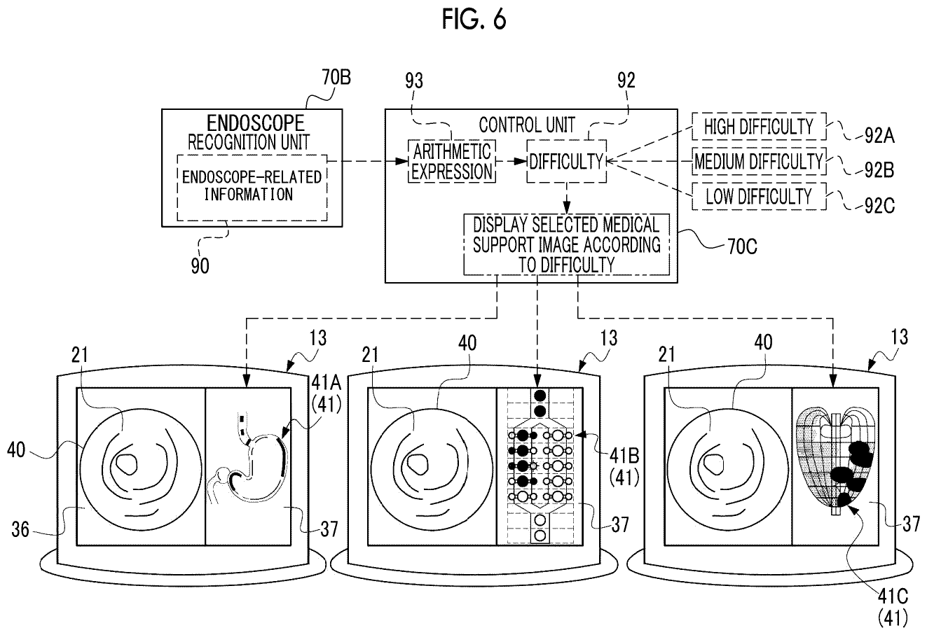

For example, as illustrated in FIG. 6, the control unit 70C acquires the endoscope-related information 90 from the endoscope recognition unit 70B and calculates difficulty 92 on the basis of the acquired endoscope-related information 90. The difficulty 92 indicates the difficulty of the technique using the endoscope 12 and/or the difficulty of the mental rotation. The difficulty 92 is calculated from an arithmetic expression 93. The arithmetic expression 93 is an arithmetic expression that has numerical values indicating the information included in the endoscope-related information 90 (for example, a numerical value indicating the treatment tool information 90A, a numerical value indicating the operation speed information 90B, a numerical value indicating the positional information 90C, a numerical value indicating the shape information 90D, and a numerical value indicating the fluid delivery information 90E) as independent variables and has the difficulty 92 as a dependent variable.

The difficulty 92 is roughly classified into, for example, three levels of high difficulty 92A, medium difficulty 92B, and low difficulty 92C. That is, the control unit 70C calculates any one of the high difficulty 92A, the medium difficulty 92B, or the low difficulty 92C from the arithmetic expression 93.

The control unit 70C displays the medical support image 41 on the screen 37 of the display device 13. The medical support image 41 is classified into a first medical support image 41A, a second medical support image 41B, and a third medical support image 41C. The first medical support image 41A, the second medical support image 41B, and the third medical support image 41C are an example of “a plurality of images” and “a plurality of schematic views” according to the technology of the present disclosure. The first medical support image 41A is an example of a “second schematic view” and a “second image” according to the technology of the present disclosure. The second medical support image 41B is an example of a “first schematic view”, the “second image”, and a “fourth schematic view” according to the technology of the present disclosure. The third medical support image 41C is an example of a “third schematic view”, the “second image”, and a “fifth schematic view” according to the technology of the present disclosure.

The amount of visual information of the first medical support image 41A, the amount of visual information of the second medical support image 41B, and the amount of visual information of the third medical support image 41C are different from one another. The first medical support image 41A has a smaller amount of information than the second medical support image 41B and the third medical support image 41C, and the second medical support image 41B has a smaller amount of information than the third medical support image 41C. In other words, the first medical support image 41A is an image in a simple format, and the second medical support image 41B and the third medical support image 41C are images in a more detailed format than the first medical support image 41A. In addition, the third medical support image 41C is an image in a more detailed format than the second medical support image 41B.

Here, in the relationship between the first medical support image 41A and the second and third medical support images 41B and 41C, the amount of information of the first medical support image 41A is a “second amount of information” according to the technology of the present disclosure, and the amount of information of the second medical support image 41B and the amount of information of the third medical support image 41C are examples of a “first amount of information” according to the technology of the present disclosure. In the relationship between the second medical support image 41B and the third medical support image 41C, the amount of information of the second medical support image 41B is an example of the “second amount of information” according to the technology of the present disclosure, and the amount of information of the third medical support image 41C is an example of the “first amount of information” according to the technology of the present disclosure.

In the relationship between the first medical support image 41A and the second and third medical support images 41B and 41C, the first medical support image 41A is a “simple image” and a “third image” according to the technology of the present disclosure, and the second medical support image 41B and the third medical support image 41C are examples of a “detailed image” and “at least one fourth image” according to the technology of the present disclosure. In the relationship between the second medical support image 41B and the third medical support image 41C, the second medical support image 41B is an example of the “simple image” and the “third image” according to the technology of the present disclosure, and the third medical support image 41C is an example of the “detailed image” and the “fourth image” according to the technology of the present disclosure.

The control unit 70C displays the first medical support image 41A as a default medical support image 41 on the screen 37. Then, the control unit 70C selectively displays the first medical support image 41A, the second medical support image 41B, and the third medical support image 41C on the screen 37, using the first medical support image 41A as a starting point.

The high difficulty 92A is associated with the first medical support image 41A. The medium difficulty 92B is associated with the second medical support image 41B. The low difficulty 92C is associated with the third medical support image 41C.

The control unit 70C selects the first medical support image 41A, the second medical support image 41B, and the third medical support image 41C according to the difficulty 92 calculated from the arithmetic expression 93 and displays the selected medical support image 41 on the screen 37. In other words, the control unit 70C performs the switching among the first medical support image 41A, the second medical support image 41B, and the third medical support image 41C as the medical support images 41 to be displayed on the screen 37 according to the difficulty 92 calculated on the basis of the information included in the endoscope-related information 90.

In this case, for example, in a case in which the high difficulty 92A is calculated from the arithmetic expression 93, the first medical support image 41A is displayed on the screen 37. In addition, in a case in which the medium difficulty 92B is calculated from the arithmetic expression 93, the second medical support image 41B is displayed on the screen 37. Further, in a case in which the low difficulty 92C is calculated from the arithmetic expression 93, the third medical support image 41C is displayed on the screen 37.

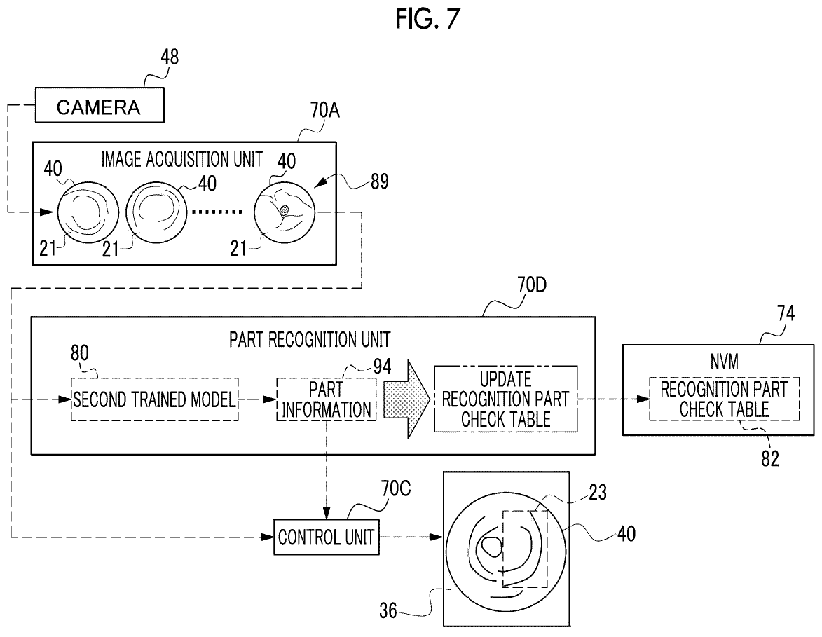

For example, as illustrated in FIG. 7, the part recognition unit 70D performs the image recognition process using the second trained model 80 on the time-series image group 89 (that is, a plurality of time-series endoscopic images 40 held by the image acquisition unit 70A) to recognize a part of the observation target 21. In other words, the recognition of the part can be said to be the detection of the part. In this embodiment, the recognition of the part indicates a process that specifies the name of the part and stores the endoscopic image 40 including the recognized part and the name of the part included in the endoscopic image 40 in a memory (for example, the NVM74 and/or an external storage device) to be associated with each other.

The second trained model 80 is obtained by performing machine learning using second training data on the neural network to optimize the neural network. An example of the second training data is training data in which a plurality of images (for example, a plurality of images corresponding to a plurality of time-series endoscopic images 40) obtained in time series by imaging a part (for example, a part in the observation target 21) to be subjected to endoscopy are example data and part information 94 related to the part to be subjected to endoscopy is correct answer data. There are a plurality of parts such as a cardia, a vault, a greater-curvature-side anterior wall of an upper gastric body, a greater-curvature-side posterior wall of the upper gastric body, a greater-curvature-side anterior wall of a middle gastric body, a greater-curvature-side posterior wall of the middle gastric body, a greater-curvature-side anterior wall of a lower gastric body, and a greater-curvature-side posterior wall of the lower gastric body. Machine learning is performed on the neural network using the second training data created for each part. The part information 94 includes, for example, information indicating the name of the part, coordinates that can specify the position of the part in the observation target 21.

In addition, here, an example of the form in which only one second trained model 80 is used by the part recognition unit 70D has been described. However, this is only an example. For example, the second trained model 80 selected from a plurality of second trained models 80 may be used by the part recognition unit 70D. In this case, each of the second trained models 80 may be created by performing machine learning specialized for each type of endoscopy. The second trained model 80 corresponding to the type of endoscopy that is currently being performed may be selected and used by the part recognition unit 70D.

In this embodiment, a trained model created by performing machine learning specialized for endoscopy for the stomach is applied as an example of the second trained model 80 used by the part recognition unit 70D.

In addition, here, an example of the form in which the second trained model 80 is created by performing machine learning specialized for endoscopy for the stomach on the neural network has been described. However, this is only an example. In a case in which endoscopy is performed for a luminal organ other than the stomach, a trained model created by performing machine learning specialized for the type of luminal organ to be subjected to endoscopy on the neural network may be used as the second trained model 80. An example of the luminal organ other than the stomach is the large intestine, the small intestine, the esophagus, the duodenum, or the bronchus. In addition, a trained model created by performing machine learning specialized for endoscopy for a plurality of luminal organs, such as the stomach, the large intestine, the small intestine, the esophagus, the duodenum, and the bronchus, on the neural network may be used as the second trained model 80.

The part recognition unit 70D performs the image recognition process using the second trained model 80 on the time-series image group 89 acquired by the image acquisition unit 70A to recognize a plurality of parts included in the stomach (hereinafter, simply referred to as “a plurality of parts”). The plurality of parts are classified into major categories and minor categories included in the major categories. The “major category” referred to here is an example of a “major category” according to the technology of the present disclosure. In addition, the “minor category” referred to here is an example of a “minor category” according to the technology of the present disclosure.

The plurality of parts are roughly classified into the cardia, the vault, the greater curvature of the upper gastric body, the greater curvature of the middle gastric body, the greater curvature of the lower gastric body, the greater curvature of the gastric angle, the greater curvature of the antrum, the duodenal bulb, the pyloric ring, the lesser curvature of the antrum, the lesser curvature of the gastric angle, the lesser curvature of the upper gastric body, the lesser curvature of the middle gastric body, and the lesser curvature of the lower gastric body as the major categories.

The greater curvature of the upper gastric body is classified into the greater-curvature-side anterior wall of the upper gastric body and the greater-curvature-side posterior wall of the upper gastric body as the minor categories. The greater curvature of the middle gastric body is classified into the greater-curvature-side anterior wall of the middle gastric body and the greater-curvature-side posterior wall of the middle gastric body as the minor categories. The greater curvature of the lower gastric body is classified into the greater-curvature-side anterior wall of the lower gastric body and the greater-curvature-side posterior wall of the lower gastric body as the minor categories. The greater curvature of the gastric angle is classified into the greater-curvature-side anterior wall of the gastric angle and the greater-curvature-side posterior wall of the gastric angle as the minor categories. The greater curvature of the antrum is classified into the greater-curvature-side anterior wall of the antrum and the greater-curvature-side posterior wall of the antrum as the minor categories. The lesser curvature of the antrum is classified into the lesser-curvature-side anterior wall of the antrum and the lesser-curvature-side posterior wall of the antrum as the minor categories. The lesser curvature of the gastric angle is classified into the lesser-curvature-side anterior wall of the gastric angle and the lesser-curvature-side posterior wall of the gastric angle as the minor categories. The lesser curvature of the lower gastric body is classified into the lesser-curvature-side anterior wall of the lower gastric body and the lesser-curvature-side posterior wall of the lower gastric body as the minor categories. The lesser curvature of the middle gastric body is classified into the lesser-curvature-side anterior wall of the middle gastric body and the lesser-curvature-side posterior wall of the middle gastric body as the minor categories. The lesser curvature of the upper gastric body is classified into the lesser-curvature-side anterior wall of the upper gastric body and the lesser-curvature-side posterior wall of the upper gastric body as the minor categories.

The part recognition unit 70D acquires the time-series image group 89 from the image acquisition unit 70A and inputs the acquired time-series image group 89 to the second trained model 80. Then, the second trained model 80 outputs the part information 94 corresponding to the input time-series image group 89. The part recognition unit 70D acquires the part information 94 output from the second trained model 80.

The recognition part check table 82 is a table that is used to check whether or not the part scheduled to be recognized by the part recognition unit 70D has been recognized. In the recognition part check table 82, the plurality of parts are associated with information indicating whether or not each part has been recognized by the part recognition unit 70D. Since the name of the part is specified from the part information 94, the part recognition unit 70D updates the recognition part check table 82 according to the part information 94 acquired from the second trained model 80. That is, the part recognition unit 70D updates the information corresponding to each part in the recognition part check table 82 (that is, the information indicating whether or not the part has been recognized by the part recognition unit 70D).

The control unit 70C displays the endoscopic image 40 acquired by the image acquisition unit 70A on the screen 36. The control unit 70C generates a detection frame 23 on the basis of the part information 94 and displays the generated detection frame 23 to be superimposed on the endoscopic image 40. The detection frame 23 is a frame that can specify the position of the part specified from the part information 94. For example, the detection frame 23 is generated on the basis of a bounding box that is used in the AI-type image recognition process. The detection frame 23 may be a rectangular frame that consists of a continuous line or a frame having a shape other than the rectangular shape. Further, for example, instead of the rectangular frame consisting of the continuous line, a frame that consists of discontinuous lines (that is, intermittent lines) may be used. In addition, for example, a plurality of marks that specify portions corresponding to four corners of the detection frame 23 may be displayed. Further, the part specified from the part information 94 may be filled with a predetermined color (for example, a translucent color).

Furthermore, here, an example of the form in which the AI-type process (for example, the process by the endoscope recognition unit 70B and the process by the part recognition unit 70D) is performed by the control device 22 has been described. However, the technology of the present disclosure is not limited thereto. For example, the AI-type process may be performed by a device that is separate from the control device 22. In this case, for example, the device that is separate from the control device 22 acquires the endoscopic image 40 and various parameters used to observe the observation target 21 with the endoscope 12 and outputs an image obtained by superimposing the detection frame 23 and/or various maps (for example, the medical support image 41) on the endoscopic image 40 to the display device 13 and the like.

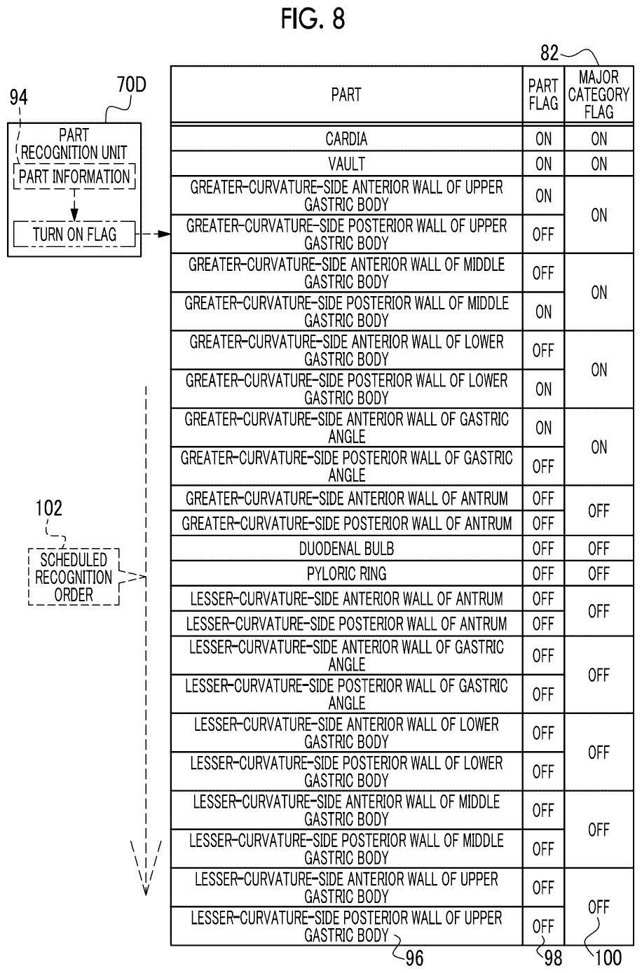

For example, as illustrated in FIG. 8, the recognition part check table 82 is a table in which a part name 96 is associated with a part flag 98 and a major category flag 100. The part name 96 is the name of a part. In the recognition part check table 82, a plurality of part names 96 are arranged in a scheduled recognition order 102. The scheduled recognition order 102 indicates the order of parts scheduled to be recognized by the part recognition unit 70D.

The part flag 98 is a flag indicating whether or not the part corresponding to the part name 96 has been recognized by the part recognition unit 70D. The part flag 98 is switched between on (for example, 1) and off (for example, 0). The part flag 98 is off as a default. In a case in which the part corresponding to the part name 96 is recognized, the part recognition unit 70D turns on the part flag 98 corresponding to the part name 96 indicating the recognized part.

The major category flag 100 is a flag indicating whether or not the part corresponding to the major category has been recognized by the part recognition unit 70D. The major category flag 100 is switched between on (for example, 1) and off (for example, 0). The major category flag 100 is off as a default. In a case in which the part recognition unit 70D recognizes a part classified into the major category (for example, a part classified into the minor category among the parts classified into the major category), that is, a part corresponding to the part name 96, the major category flag 100 corresponding to the major category into which the recognized portion is classified is turned on. In other words, in a case in which the part flag 98 corresponding to the major category flag 100 is turned on, the major category flag 100 is turned on.

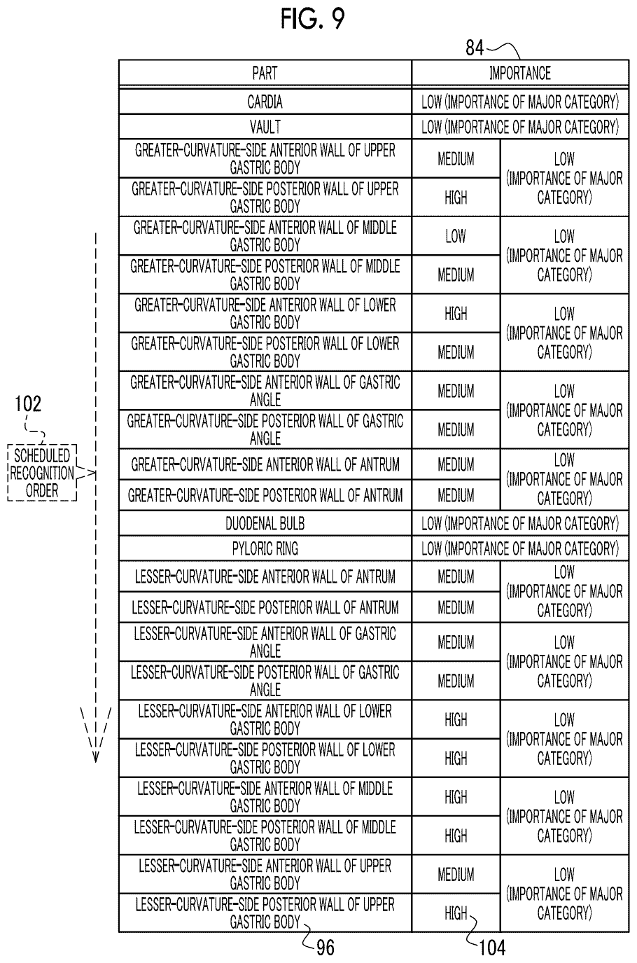

For example, as illustrated in FIG. 9, the importance table 84 is a table in which importance 104 is associated with the part name 96. That is, the importance 104 is given to a plurality of parts.

In the importance table 84, a plurality of part names 96 are arranged in the order of the parts scheduled to be recognized by the part recognition unit 70D. That is, in the importance table 84, the plurality of part names 96 are arranged in the scheduled recognition order 102. The importance 104 is the importance of the part specified from the part name 96. The importance 104 is defined by any one of three levels of a “high” level, a “medium” level, and a “low” level. The “high” level or the “medium” level is given as the importance 104 to the part classified into the minor category, and the “low” level is given as the importance 104 to the part classified into the major category.

In the example illustrated in FIG. 9, the “high” level is given as the importance 104 to the greater-curvature-side posterior wall of the upper gastric body, the greater-curvature-side anterior wall of the lower gastric body, the lesser-curvature-side anterior wall of the lower gastric body, the lesser-curvature-side posterior wall of the lower gastric body, the lesser-curvature-side anterior wall of the middle gastric body, the lesser-curvature-side posterior wall of the middle gastric body, and the lesser-curvature-side posterior wall of the upper gastric body.

The “medium” level is given as the importance 104 to each part classified into the minor category other than the greater-curvature-side posterior wall of the upper gastric body, the greater-curvature-side anterior wall of the middle gastric body, the greater-curvature-side anterior wall of the lower gastric body, the lesser-curvature-side anterior wall of the lower gastric body, the lesser-curvature-side posterior wall of the lower gastric body, the lesser-curvature-side anterior wall of the middle gastric body, the lesser-curvature-side posterior wall of the middle gastric body, and the lesser-curvature-side posterior wall of the upper gastric body. That is, the “medium” level is given as the importance 104 to the greater-curvature-side anterior wall of the upper gastric body, the greater-curvature-side posterior wall of the middle gastric body, the greater-curvature-side posterior wall of the lower gastric body, the greater-curvature-side anterior wall of the gastric angle, the greater-curvature-side posterior wall of the gastric angle, the greater-curvature-side anterior wall of the antrum, the greater-curvature-side posterior wall of the antrum, the lesser-curvature-side anterior wall of the antrum, the lesser-curvature-side posterior wall of the antrum, the lesser-curvature-side anterior wall of the gastric angle, the lesser-curvature-side posterior wall of the gastric angle, and the lesser-curvature-side anterior wall of the upper gastric body.

In addition, in the example illustrated in FIG. 9, the “low” level is given as the importance 104 to the greater-curvature-side anterior wall of the middle gastric body, the cardia, the vault, the greater curvature of the upper gastric body, the greater curvature of the middle gastric body, the greater curvature of the lower gastric body, the greater curvature of the gastric angle, the greater curvature of the antrum, the duodenal bulb, the pyloric ring, the lesser curvature of the antrum, the lesser curvature of the gastric angle, the lesser curvature of the upper gastric body, the lesser curvature of the middle gastric body, and the lesser curvature of the lower gastric body.

In addition, here, for convenience of explanation, the “low” level is given as the importance 104 to the greater-curvature-side anterior wall of the middle gastric body. However, this is only an example. For example, the importance 104 of each of the parts classified into the major categories, such as the cardia, the vault, the greater curvature of the upper gastric body, the greater curvature of the middle gastric body, the greater curvature of the lower gastric body, the greater curvature of the gastric angle, the greater curvature of the antrum, the duodenal bulb, the pyloric ring, the lesser curvature of the antrum, the lesser curvature of the gastric angle, the lesser curvature of the lower gastric body, the lesser curvature of the middle gastric body, and the lesser curvature of the upper gastric body, may be lower than that of the part classified into the minor category. In other words, the part classified into the minor category may be given higher importance 104 than the part classified into the major category.

The “high”, “medium”, and “low” levels of the importance 104 are determined in response to an instruction given from the outside to the endoscope 12. The receiving device 62 is given as an example of a first unit that gives an instruction for the importance 104 to the endoscope 12. In addition, a communication device (for example, a tablet terminal, a personal computer, and/or a server) that is connected to the endoscope 12 such that it can communicate therewith) is given as an example of a second unit that gives an instruction for the importance 104 to the endoscope 12.

In addition, the importance 104 associated with the plurality of part names 96 is determined according to the data of the past examination (for example, statistical data based on the data of the past examination obtained from a plurality of subjects 20) performed on a plurality of parts.

For example, the importance 104 corresponding to a part, which is determined to be a part for which the omission of recognition is typically likely to occur, among a plurality of parts is set to be higher than the importance 104 corresponding to a part, which is determined to be a part for which the omission of recognition is typically unlikely to occur, among the plurality of parts. Whether or not the omission of recognition is typically likely to occur is derived from the data of the past examination performed on a plurality of parts by, for example, a statistical method. In this embodiment, the “high” importance 104 indicates that the possibility that the omission of recognition will typically occur is high. In addition, the “medium” importance 104 indicates that the possibility that the omission of recognition will typically occur is medium. In addition, the “low” importance 104 indicates that the possibility that the omission of recognition will typically occur is low.

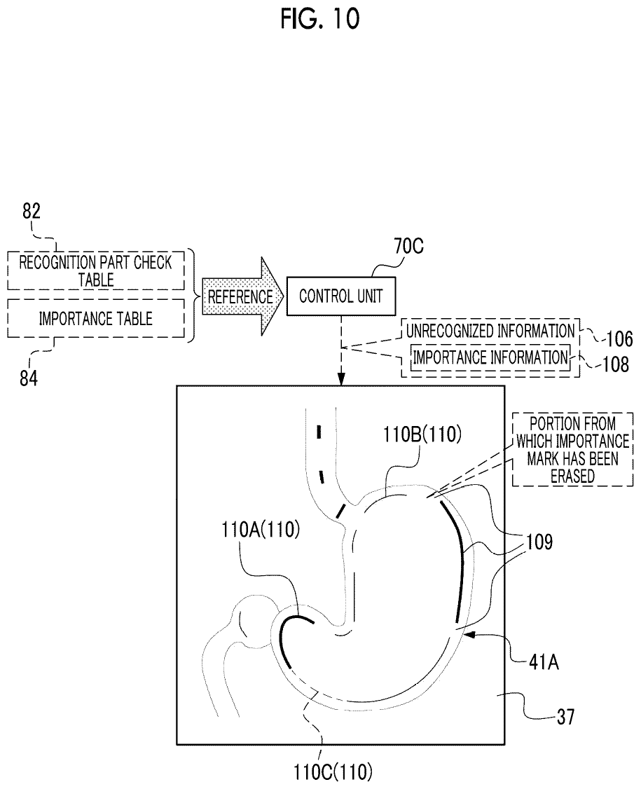

For example, as illustrated in FIG. 10, the control unit 70C outputs unrecognized information 106 in a case in which an unrecognized part (that is, a part that has not been recognized by the part recognition unit 70D) of the observation target 21 is present among a plurality of parts according to the recognition part check table 82 and the importance table 84. In this embodiment, the unrecognized information 106 is output in a case in which it is confirmed that an unrecognized part (that is, an unobserved part) of the observation target 21 is present among a plurality of parts. The unrecognized information 106 is information that can specify that the unrecognized part is present. In other words, the unrecognized information 106 is information indicating that an unobserved part (that is, a part that has not been observed) is present among a plurality of parts. The unrecognized information 106 is an example of “unobserved information” according to the technology of the present disclosure. The unrecognized information 106 includes importance information 108. The importance information 108 is information that can specify the importance 104 obtained from the importance table 84.

The output destination of the unrecognized information 106 is the display device 13. However, this is only an example, and the output destination of the unrecognized information 106 may be, for example, a tablet terminal, a personal computer, and/or a server that is connected to the endoscope 12 such that it can communicate therewith.

In a case in which the control unit 70C selects the first medical support image 41A as the medical support image 41 to be displayed on the screen 37 in the above-described manner, the control unit 70C displays the unrecognized information 106 as the first medical support image 41A on the screen 37. The control unit 70C displays the importance information 108 included in the unrecognized information 106 as an importance mark 110 in the first medical support image 41A.

The first medical support image 41A is a schematic perspective view showing a schematic aspect of the stomach. In addition, the first medical support image 41A is divided into a plurality of regions 109 corresponding to a plurality of parts of the observation target 21 observed through the endoscope 12 and is represented in an aspect different from that in which the second medical support image 41B and the third medical support image 41C are represented. The first medical support image 41A is divided into the plurality of regions 109 for each major category and each minor category. In the example illustrated in FIG. 10, the plurality of regions 109 are linearly divided according to the shape of the stomach inside the outline of the stomach shown by the first medical support image 41A. In addition, the plurality of regions 109 may be classified into only the major categories or may be classified into only the minor categories.

The display aspect of the importance mark 110 differs depending on the importance information 108. The importance mark 110 is classified into a first importance mark 110A, a second importance mark 110B, and a third importance mark 110C. The first importance mark 110A is a mark indicating the “high” importance 104. The second importance mark 110B is a mark indicating the “medium” importance 104. The third importance mark 110C is a mark indicating the “low” importance 104. That is, the first importance mark 110A, the second importance mark 110B, and the third importance mark 110C are marks that are represented in a display aspect in which the “high”, “medium”, and “low” levels of importance can be distinguished. The second importance mark 110B is displayed in a state in which it is emphasized more than the third importance mark 110C, and the first importance mark 110A is displayed in a state in which it is emphasized more than the second importance mark 110B. In the example illustrated in FIG. 10, the thickness of the line of the second importance mark 110B is larger than the thickness of the line of the third importance mark 110C, and the thickness of the line of the first importance mark 110A is larger than the thickness of the line of the second importance mark 110B.

In the first medical support image 41A, the importance mark 110 corresponding to the importance information 108 is displayed to be superimposed on the region 109 corresponding to the part which has not been recognized by the part recognition unit 70D. In a case in which the part recognition unit 70D recognizes the part corresponding to the region 109 on which the importance mark 110 is displayed to be superimposed in the first medical support image 41A, the importance mark 110 displayed to be superimposed on the region 109 corresponding to the recognized part is erased. Therefore, in the first medical support image 41A, the plurality of regions 109 are classified into a first observed region and a second unobserved region. The first observed region is an example of an “observed region” according to the technology of the present disclosure, and the first unobserved region is an example of an “unobserved region” according to the technology of the present disclosure.

The first observed region indicates a region corresponding to the part observed by the doctor 14 in the first medical support image 41A, that is, the region 109 corresponding to the part recognized by the part recognition unit 70D. The first unobserved region indicates a region corresponding to the part which has not been observed by the doctor 14 in the first medical support image 41A, that is, the region 109 corresponding to the part which has not been recognized by the part recognition unit 70D.

The first observed region is the region 109 on which the importance mark 110 is displayed to be superimposed in the first medical support image 41A, and the first unobserved region is the region 109 on which the importance mark 110 is not displayed to be superimposed in the first medical support image 41A. As a result, in the first medical support image 41A, the first observed region is displayed to be emphasized more than the first unobserved region. Therefore, the doctor 14 can visually understand for which part the omission of recognition has occurred.

The control unit 70C updates the content of the first medical support image 41A in a case in which the major category flag 100 in the recognition part check table 82 is turned on. The update of the content of the first medical support image 41A is achieved by the output of the unrecognized information 106 by the control unit 70C.

In a case in which the major category flag 100 in the recognition part check table 82 is turned on, the control unit 70C fills the region 109 corresponding to the turned-on major category flag 100 with the same color as a background color. In addition, in a case in which the part flag 98 is turned on, the control unit 70C fills the region corresponding to the turned-on part flag 98 with the same color as the background color.

Further, in a case in which a plurality of minor categories are included in the major category and the part flag 98 corresponding to the part classified into one minor category is turned on, the major category flag 100 corresponding to the part which is classified into the minor category having the turned-on part flag 98 is turned on.

On the other hand, in a case in which a part has not been recognized by the part recognition unit 70D, the control unit 70C displays the importance mark 110 to be superimposed on the region 109 corresponding to the part which has not been recognized by the part recognition unit 70D on condition that the part recognition unit 70D recognizes a subsequent part scheduled to be recognized by the part recognition unit 70D after the part which has not been recognized by the part recognition unit 70D. That is, in a case in which it is confirmed that the order of the parts recognized by the part recognition unit 70D deviates from the scheduled recognition order 102 (FIGS. 8 and 9), the control unit 70C displays the importance mark 110 to be superimposed on the region 109 corresponding to the part which has not been recognized by the part recognition unit 70D. The reason for doing so is to notify of the omission of recognition by the part recognition unit 70D at the time when the omission of recognition by the part recognition unit 70D is confirmed (for example, the time when the possibility that the doctor 14 will forget to observe a part during the process of operating the endoscope 12 is extremely high).

Here, an example of the subsequent part that is scheduled to be recognized after the part which has not been recognized by the part recognition unit 70D is a part that is classified into the major category scheduled to be recognized immediately after the major category into which the part, which has not been recognized by the part recognition unit 70D, is classified.

For example, in a case in which the greater-curvature-side posterior wall of the upper gastric body has not been recognized by the part recognition unit 70D, the second importance mark 110B is displayed to be superimposed on the region 109 corresponding to the greater-curvature-side posterior wall of the upper gastric body on condition that the part recognition unit 70D recognizes a part that is classified into the major category scheduled to be recognized immediately after the major category into which the greater-curvature-side posterior wall of the upper gastric body is classified. Here, the major category into which the greater-curvature-side posterior wall of the upper gastric body is classified indicates the greater curvature of the upper gastric body. In addition, the major category scheduled to be recognized immediately after the major category into which the greater-curvature-side posterior wall of the upper gastric body is classified indicates the greater curvature of the middle gastric body.

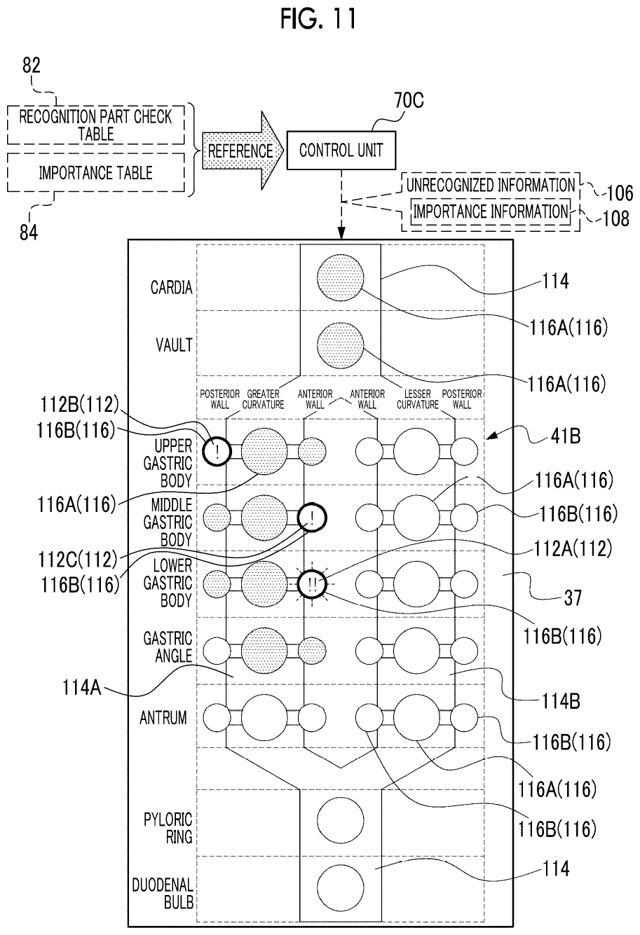

For example, as illustrated in FIG. 11, in a case in which the control unit 70C selects the second medical support image 41B as the medical support image 41 to be displayed on the screen 37 in the above-described manner, the control unit 70C displays the unrecognized information 106 as the second medical support image 41B on the screen 37. The control unit 70C displays the importance information 108 included in the unrecognized information 106 as the importance mark 112 in the second medical support image 41B.

The display aspect of the importance mark 112 differs depending on the importance information 108. The importance mark 112 is classified into a first importance mark 112A, a second importance mark 112B, and a third importance mark 112C. The first importance mark 112A is a mark representing the “high” importance 104. The second importance mark 112B is a mark representing the “medium” importance 104. The third importance mark 112C is a mark representing the “low” importance 104. That is, the first importance mark 112A, the second importance mark 112B, and the third importance mark 112C are marks that are represented in a display aspect in which the “high”, “medium”, and “small” levels of the importance can be distinguished. The second importance mark 112B is displayed in a state in which it is emphasized more than the third importance mark 112C, and the first importance mark 112A is displayed in a state in which it is emphasized more than the second importance mark 112B.

In the example illustrated in FIG. 11, the first importance mark 112A includes a plurality of exclamation marks (here, for example, two exclamation marks), and each of the second importance mark 112B and the third importance mark 112C includes one exclamation mark. The size of the exclamation mark included in the third importance mark 112C is smaller than the size of the exclamation mark included in the first importance mark 112A and the second importance mark 112B. In addition, the second importance mark 112B is colored to be more conspicuous than the third importance mark 112C, and the first importance mark 112A is colored to be more conspicuous than the second importance mark 112B. Further, the brightness of the second importance mark 112B is higher than the brightness of the third importance mark 112C, and the brightness of the first importance mark 112A is higher than the brightness of the second importance mark 112B. As described above, as a relationship of the degree of conspicuousness, a relationship of “the first importance mark 112A>the second importance mark 112B>the third importance mark 112C” is established.

The second medical support image 41B is divided into a plurality of regions corresponding to a plurality of parts of the observation target 21 observed through the endoscope 12 and is represented in an aspect different from that in which the first medical support image 41A and the third medical support image 41C are represented.

The second medical support image 41B is a schematic view showing a schematic aspect of at least one route for observing the stomach. The second medical support image 41B includes a route 114. The route 114 is a route that schematically represents the order in which the stomach is observed using the endoscope 12 (here, for example, the scheduled recognition order 102 (see FIG. 8 and FIG. 9)) and is a schematic view in which the observation target 21 is divided into a plurality of regions corresponding to a plurality of parts. In the example illustrated in FIG. 11, as an example of the plurality of regions, in the medical support image 41, the cardia, the vault, the upper gastric body, the middle gastric body, the lower gastric body, the gastric angle, the antrum, the pyloric ring, and the duodenal bulb are displayed in text, and the route 114 is also divided into the cardia, the vault, the upper gastric body, the middle gastric body, the lower gastric body, the gastric angle, the antrum, the pyloric ring, and the duodenal bulb.

The route 114 is branched into a greater-curvature-side route 114A and a lesser-curvature-side route 114B in the middle from the most upstream side to the downstream side of the stomach, and the branched routes are joined. On the route 114, a large circular mark 116A is assigned to the part classified into the major category, and a small circular mark 116B is assigned to the part classified into the minor category. That is, the second medical support image 41B is divided by a plurality of circular marks 116A for each major category and is divided by a plurality of circular marks 116B for each minor category. Hereinafter, for convenience of explanation, in a case in which the circular marks 116A and 116B do not need to be distinguished from each other for description, they are referred to as “circular marks 116”.

The second medical support image 41B is divided by a plurality of circular marks 116 disposed along the route 114. The plurality of circular marks 116 disposed along the route 114 is an example of “a plurality of regions” according to the technology of the present disclosure. In addition, a plurality of regions obtained by dividing the second medical support image 41B into the cardia, the vault, the upper gastric body, the middle gastric body, the lower gastric body, the gastric angle, the antrum, the pyloric ring, and the duodenal bulb are also an example of “the plurality of regions” according to the technology of the present disclosure.

In a portion of the route 114 from the most upstream side of the stomach to the front of the branch point of the greater-curvature-side route 114A and the lesser-curvature-side route 114B, the circular mark 116A corresponding to the cardia and the circular mark 116A corresponding to the vault are arranged from the most upstream side of the stomach to the downstream side of the stomach.

In the greater-curvature-side route 114A, the circular mark 116A corresponding to the greater curvature, the circular mark 116B corresponding to the anterior wall, and the circular mark 116B corresponding to the posterior wall are disposed in units of the parts classified into the major categories. The circular mark 116A corresponding to the greater curvature is located at the center of the greater-curvature-side route 114A, and the circular mark 116B corresponding to the anterior wall and the circular mark 116B corresponding to the posterior wall are located on the left and right sides of the circular mark 116A corresponding to the greater curvature.

In the lesser-curvature-side route 114B, the circular mark 116A corresponding to the lesser curvature, the circular mark 116B corresponding to the anterior wall, and the circular mark 116B corresponding to the posterior wall are disposed in units of the parts classified into the major categories. The circular mark 116A corresponding to the lesser curvature is located at the center of the lesser-curvature-side route 114B, and the circular mark 116B corresponding to the anterior wall and the circular mark 116B corresponding to the posterior wall are located on the left and right sides of the circular mark 116A corresponding to the lesser curvature.

In a portion of the routes 114 from a junction point of the greater-curvature-side route 114A and the lesser-curvature-side route 114B to the most downstream side of the stomach, the circular mark 116A corresponding to the pyloric ring and the circular mark 116A corresponding to the duodenal bulb are arranged.

The inside of the circular mark 116 is blank as a default. In a case in which the part corresponding to the circular mark 116 has been recognized by the part recognition unit 70D, the inside of the circular mark 116 corresponding to the part recognized by the part recognition unit 70D is filled with a specific color (for example, a predetermined color among three primary colors of light and three primary colors of color). On the other hand, in a case in which the part corresponding to the circular mark 116 has not been recognized by the part recognition unit 70D, the inside of the circular mark 116 corresponding to the part which has not been recognized by the part recognition unit 70D is not filled with any color. However, the importance mark 112 corresponding to the importance 104 of the part which has not been recognized by the part recognition unit 70D is displayed in the circular mark 116 corresponding to the part which has not been recognized by the part recognition unit 70D.

Therefore, in the second medical support image 41B, a plurality of circular marks 116 are classified into a second observed region and a second unobserved region. The second observed region indicates the circular mark 116 corresponding to the part recognized by the part recognition unit 70D, that is, the circular mark 116 filled with a specific color. The second unobserved region indicates the circular mark 116 in which the importance mark 112 is displayed. The second observed region is an example of the “observed region” according to the technology of the present disclosure, and the second unobserved region is an example of the “unobserved region” according to the technology of the present disclosure. The circular mark 116 corresponding to the part recognized by the part recognition unit 70D and the circular mark 116 corresponding to the part, which has not been recognized by the part recognition unit 70D, are displayed in the second medical support image 41B on the display device 13 in an aspect in which the circular marks 116 can be distinguished from each other. In a case in which the first medical support image 41A and the second medical support image 41B displayed on the screen 37 of the display device 13 are compared, the second observed region and the second unobserved region of the second medical support image 41B are displayed to be distinguishable in more detail than the first observed region and the first unobserved region of the first medical support image 41A.

The control unit 70C updates the content of the medical support image 41 in a case in which the major category flag 100 in the recognition part check table 82 is turned on. The update of the content of the medical support image 41 is achieved by the output of the unrecognized information 106 by the control unit 70C.

In a case in which the major category flag 100 in the recognition part check table 82 is turned on, the control unit 70C fills the circular mark 116A of the part corresponding to the turned-on major category flag 100 with a specific color. In addition, in a case in which the part flag 98 is turned on, the control unit 70C fills the circular mark 116B of the part corresponding to the turned-on part flag 98 with a specific color.

On the other hand, in a case in which a part has not been recognized by the part recognition unit 70D, the control unit 70C displays the importance mark 112 in the circular mark 116 corresponding to the part which has not been recognized by the part recognition unit 70D on condition that the part recognition unit 70D recognizes the subsequent part scheduled to be recognized by the part recognition unit 70D after the part which has not been recognized by the part recognition unit 70D. That is, in a case in which it is confirmed that the order of the parts recognized by the part recognition unit 70D deviates from the scheduled recognition order 102 (FIGS. 8 and 9), the control unit 70C displays the importance mark 112 in the circular mark 116 corresponding to the part which has not been recognized by the part recognition unit 70D.

In the example illustrated in FIG. 11, in a case in which the greater-curvature-side posterior wall of the upper gastric body has not been recognized by the part recognition unit 70D, the second importance mark 112B is displayed to be superimposed on the circular mark 116B corresponding to the greater-curvature-side posterior wall of the upper gastric body on condition that the part recognition unit 70D recognizes a part that is classified into the major category scheduled to be recognized immediately after the major category into which the greater-curvature-side posterior wall of the upper gastric body is classified. Here, the major category into which the greater-curvature-side posterior wall of the upper gastric body is classified indicates the greater curvature of the upper gastric body. In addition, the major category scheduled to be recognized immediately after the major category into which the greater-curvature-side posterior wall of the upper gastric body is classified indicates the greater curvature of the middle gastric body.

In addition, in the example illustrated in FIG. 11, in a case in which the greater-curvature-side anterior wall of the middle gastric body has not been recognized by the part recognition unit 70D, the third importance mark 112C is displayed to be superimposed on the circular mark 116B corresponding to the greater-curvature-side anterior wall of the middle gastric body on condition that the part recognition unit 70D recognizes a part that is classified into the major category scheduled to be recognized immediately after the major category into which the greater-curvature-side anterior wall of the middle gastric body is classified. Here, the major category into which greater-curvature-side anterior wall of the middle gastric body is classified indicates the greater curvature of the middle gastric body. In addition, the major category scheduled to be recognized immediately after the major category into which the greater-curvature-side anterior wall of the middle gastric body is classified indicates the greater curvature of the lower gastric body.

Further, in the example illustrated in FIG. 11, in a case in which the greater-curvature-side anterior wall of the lower gastric body has not been recognized by the part recognition unit 70D, the first importance mark 112A is displayed to be superimposed on the circular mark 116B corresponding to the greater-curvature-side anterior wall of the lower gastric body on condition that the part recognition unit 70D recognizes a part that is classified into the major category scheduled to be recognized immediately after the major category into which the greater-curvature-side anterior wall of the lower gastric body is classified. Here, the major category into which the greater-curvature-side anterior wall of the lower gastric body is classified indicates the greater curvature of the lower gastric body. In addition, the major category scheduled to be recognized immediately after the major category into which the greater-curvature-side anterior wall of the lower gastric body is classified indicates the greater curvature of the gastric angle.

In this embodiment, the image obtained by superimposing the importance mark 112 on the circular mark 116 is displayed in a state in which it is emphasized more than the image obtained by filling the circular mark 116 with a specific color in order to facilitate the specification of the part which has not been recognized by the part recognition unit 70D. In the example illustrated in FIG. 11, the edge of the image obtained by superimposing the importance mark 112 on the circular mark 116 is displayed in a state in which it is enhanced more than the edge of the image obtained by filling the circular mark 116 with a specific color. The enhancement of the edge is achieved, for example, by adjusting the brightness of the edge. In addition, while the image obtained by filling the circular mark 116 with a specific color does not include the exclamation mark, the image obtained by superimposing the importance mark 112 on the circular mark 116 includes the exclamation mark. Therefore, the part not recognized by the part recognition unit 70D and the part recognized by the part recognition unit 70D are visually specified depending on whether or not the exclamation mark is present.

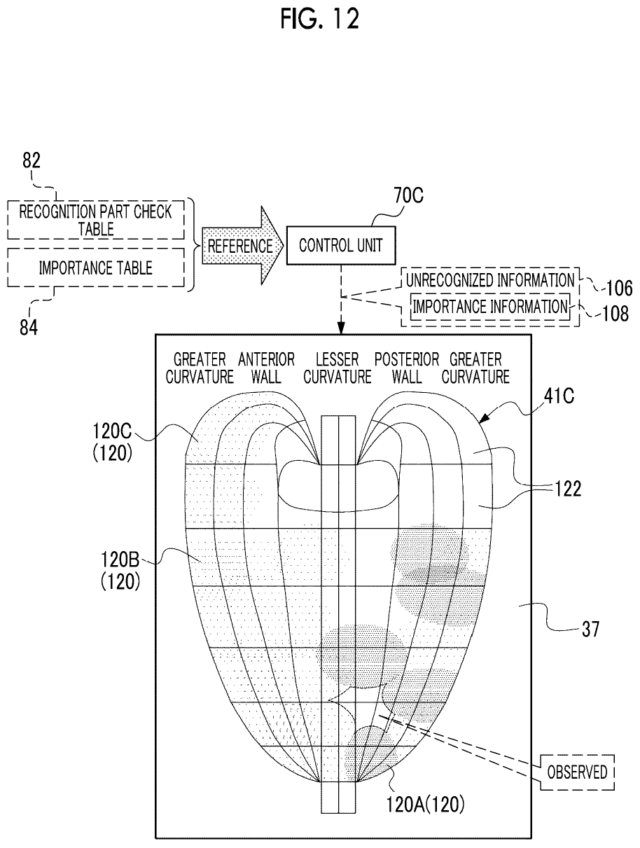

For example, as illustrated in FIG. 12, in a case in which the control unit 70C selects the third medical support image 41C as the medical support image 41 to be displayed on the screen 37 in the above-described manner, the control unit 70C displays the unrecognized information 106 as the third medical support image 41C on the screen 37. The control unit 70C displays the importance information 108 included in the unrecognized information 106 as an importance mark 120 in the third medical support image 41C. The third medical support image 41C is a schematic view showing an aspect in which the stomach is schematically developed. The third medical support image 41C is divided into a plurality of regions 122 for each major category and each minor category. The importance marks 120 are elliptical marks and are distributed at positions corresponding to the plurality of regions 122 in the third medical support image 41C. In addition, the plurality of regions 122 may be classified into only the major categories or only the minor categories.

The display aspect of the importance mark 120 differs depending on the importance information 108. The importance marks 120 are classified into a first importance mark 120A, a second importance mark 120B, and a third importance mark 120C. The first importance mark 120A is a mark representing “high” importance 104. The second importance mark 120B is a mark representing “medium” importance 104. The third importance mark 120C is a mark representing “low” importance 104. That is, the first importance mark 120A, the second importance mark 120B, and the third importance mark 120C are marks that are represented in a display aspect in which the “high”, “medium”, and “low” levels of importance can be distinguished. The second importance mark 120B is displayed in a state in which it is emphasized more than the third importance mark 120C, and the first importance mark 120A is displayed in a state in which it is emphasized more than the second importance mark 120B.

The third medical support image 41C is divided into a plurality of regions 122 corresponding to a plurality of parts of the observation target 21 observed through the endoscope 12 and is represented in an aspect different from that in which the first medical support image 41A and the second medical support image 41B are represented.

The plurality of regions 122 are blank as a default. In a case in which a part corresponding to the region 122, a part corresponding to a portion of the region 122, or a part corresponding to a portion that extends over a plurality of regions 122 has been recognized by the part recognition unit 70D, the region 122 corresponding to the part recognized by the part recognition unit 70D is filled with the same color as the background color.

On the other hand, in a case in which the part corresponding to the region 122, the part corresponding to a portion of the region 122, or the part corresponding to the portion that extends over the plurality of regions 122 has not been recognized by the part recognition unit 70D, the importance mark 120 corresponding to the importance information 108 is displayed for the part which has not been recognized by the part recognition unit 70D.

Therefore, the inside of the third medical support image 41C is classified into a third observed region and a third unobserved region. The third observed region indicates a blank region corresponding to the part recognized by the part recognition unit 70D (that is, a region filled with the same color as the background color). The third unobserved region indicates a region to which the importance mark 120 corresponding to the part that has not been recognized by the part recognition unit 70D is attached. The third observed region is an example of the “observed region” according to the technology of the present disclosure, and the third unobserved region is an example of the “unobserved region” according to the technology of the present disclosure. The third medical support image 41C is divided into a region to which the importance mark 120 is attached and a region to which the importance mark 120 is not attached on the display device 13. That is, the third observed region and the third unobserved region are displayed in the third medical support image 41C in an aspect in which they can be distinguished from each other. In a case in which the first medical support image 41A and the third medical support image 41C displayed on the screen 37 of the display device 13 are compared, the third observed region and the third unobserved region of the third medical support image 41C are displayed to be distinguishable in more detail than the first observed region and the first unobserved region of the first medical support image 41A. Further, in a case in which the second medical support image 41B and the third medical support image 41C displayed on the screen 37 of the display device 13 are compared, the third observed region and the third unobserved region of the third medical support image 41C are displayed to be distinguishable in more detail than the second observed region and the second unobserved region of the second medical support image 41B.

The control unit 70C erases the importance mark 120 corresponding to the part recognized by the part recognition unit 70D from the third medical support image 41C. As a result, a portion in which the importance mark 120 is displayed in the third medical support image 41C is displayed to be emphasized more than a portion in which the importance mark 120 is not displayed (for example, a portion from which the importance mark 120 has been erased) in the third medical support image 41C. Therefore, the doctor 14 easily visually understands that the portion in which the importance mark 120 is displayed in the third medical support image 41C is a portion corresponding to the part which has not been recognized by the part recognition unit 70D and the part in which the importance mark 120 is not displayed is a portion corresponding to the part recognized by the part recognition unit 70D.

Next, the operation of a portion of the endoscope system 10 according to the technology of the present disclosure will be described with reference to FIGS. 13A and 13B.

FIGS. 13A and 13B illustrate an example of a flow of the medical support process performed by the processor 70. The flow of the medical support process illustrated in FIGS. 13A and 13B is an example of a “medical support method” according to the technology of the present disclosure.

In the medical support process illustrated in FIGS. 13A and 13B, first, in Step ST10, the control unit 70C displays the first medical support image 41A as the default medical support image 41 on the screen 37. After the process in Step ST10 is performed, the medical support process proceeds to Step ST12.

In Step ST12, the image acquisition unit 70A determines whether or not imaging corresponding to one frame has been performed by the camera 48. In a case in which the imaging corresponding to one frame has not been performed by the camera 48 in Step ST12, the determination result is “No”, and the determination in Step ST10 is performed again. In a case in which the imaging corresponding to one frame has been performed by the camera 48 in Step ST12, the determination result is “Yes”, and the medical support process proceeds to Step ST14.

In Step ST14, the image acquisition unit 70A acquires the endoscopic image 40 of one frame from the camera 48. After the process in Step ST14 is performed, the medical support process proceeds to Step ST16.

In Step ST16, the image acquisition unit 70A determines whether or not the endoscopic images 40 of a predetermined number of frames are held. In a case in which the endoscopic images 40 of the predetermined number of frames are not held in Step ST16, the determination result is “No”, and the medical support process proceeds to Step ST12. In a case in which the endoscopic images 40 of the predetermined number of frames are held in Step ST16, the determination result is “Yes”, and the medical support process proceeds to Step ST18.

In Step ST18, the image acquisition unit 70A adds the endoscopic image 40 acquired in Step ST14 to the time-series image group 89 using the FIFO method to update the time-series image group 89. After the process in Step ST18 is performed, the medical support process proceeds to Step ST20.

In Step ST20, the control unit 70C determines whether or not a condition for directing the endoscope recognition unit 70B and the part recognition unit 70D to start the image recognition process (hereinafter, referred to as an “image recognition start condition”) is satisfied. An example of the image recognition start condition is a condition that the receiving device 62 and the like receive an instruction for the endoscope recognition unit 70B and the part recognition unit 70D to start the image recognition process. An example of the instruction for the endoscope recognition unit 70B and the part recognition unit 70D to start the image recognition process is an instruction for the camera 48 to start main exposure (for example, an instruction to start imaging for still images or imaging for recording moving images). In a case in which the image recognition start condition is not satisfied in Step ST20, the determination result is “No”, and the medical support process proceeds to Step ST12. In a case in which the image recognition start condition is satisfied in Step ST20, the determination result is “Yes”, and the medical support process proceeds to Step ST22.

In Step ST22, the endoscope recognition unit 70B performs the image recognition process using the first trained model 78 on the time-series image group 89 updated in Step ST18 to acquire the endoscope-related information 90. After the process in Step ST22 is performed, the medical support process proceeds to Step ST24.

In Step ST24, the control unit 70C calculates the difficulty 92 corresponding to the endoscope-related information 90 acquired in Step ST22 using the arithmetic expression 93. After the process in Step ST24 is performed, the medical support process proceeds to Step ST26.

In Step ST26, the control unit 70C displays the medical support image 41 selected according to the difficulty 92 calculated in Step ST24 on the screen 37. That is, the control unit 70C selects the medical support image 41 corresponding to the difficulty 92 from the first medical support image 41A, the second medical support image 41B, and the third medical support image 41C and displays the selected medical support image 41 on the screen 37. After the process in Step ST26 is performed, the medical support process proceeds to Step ST28 illustrated in FIG. 13B.

In Step ST28 illustrated in FIG. 13B, the part recognition unit 70D starts the execution of the image recognition process using the second trained model 80 on the time-series image group 89 updated in Step ST18. After the process in Step ST28 is performed, the medical support process proceeds to Step ST30.

In Step ST30, the part recognition unit 70D determines whether or not any of a plurality of parts in the observation target 21 has been recognized. In a case in which the part recognition unit 70D has not recognized any of the plurality of parts in the observation target 21 in Step ST30, the determination result is “No”, and the medical support process proceeds to Step ST40. In a case in which the part recognition unit 70D has recognized any of the plurality of parts in the observation target 21 in Step ST30, the determination result is “Yes”, and the medical support process proceeds to Step ST32.

In Step ST32, the part recognition unit 70D updates the recognition part check table 82. That is, the part recognition unit 70D turns on the part flag 98 and the major category flag 100 corresponding to the recognized part to update the recognition part check table 82. After the process in Step ST32 is performed, the medical support process proceeds to Step ST34.

In Step ST34, the control unit 70C determines whether or not the omission of recognition has occurred for the part scheduled in advance to be recognized by the part recognition unit 70D. The determination of whether or not the omission of recognition has occurred is achieved, for example, by determining whether or not the order of the parts recognized by the part recognition unit 70D deviates from the scheduled recognition order 102. In a case in which the omission of recognition has occurred for the part scheduled in advance to be recognized by the part recognition unit 70D in Step ST34, the determination result is “Yes”, and the medical support process proceeds to Step ST36. In a case in which the omission of recognition has not occurred for the part scheduled in advance to be recognized by the part recognition unit 70D in Step ST34, the determination result is “No”, and the medical support process proceeds to Step ST40.

In a case in which the determination result is “No” in Step ST34 in a state in which the medical support image 41 is not displayed on the screen 37, the control unit 70C updates the content of the medical support image 41. For example, in a case in which the first medical support image 41A is displayed on the screen 37, the control unit 70C fills a region 109 which corresponds to the part recognized by the part recognition unit 70D among the plurality of regions 109 in the first medical support image 41A with the same color as the background color. In addition, in a case in which the second medical support image 41B is displayed on the screen 37, the control unit 70C fills a circular mark 116 which corresponds to the part recognized by the part recognition unit 70D among the plurality of circular marks 116 in the second medical support image 41B with a specific color. Further, in a case in which the third medical support image 41C is displayed on the screen 37, the control unit 70C fills a region 112 which corresponds to the region recognized by the part recognition unit 70D among the plurality of regions 112 with the same color as the background color.

In Step ST36, the control unit 70C determines whether or not a part subsequent to the part not recognized by the part recognition unit 70D has been recognized by the part recognition unit 70D. The part subsequent to the part not recognized by the part recognition unit 70D indicates, for example, a part that is classified into a major category scheduled to be recognized by the part recognition unit 70D immediately after the major category in which the part not recognized by the part recognition unit 70D is classified. In a case in which the part subsequent to the part not recognized by the part recognition unit 70D has not been recognized by the part recognition unit 70D in Step ST36, the determination result is “No”, and the medical support process proceeds to Step ST40. In a case in which the part subsequent to the part not recognized by the part recognition unit 70D has been recognized by the part recognition unit 70D in Step ST36, the determination result is “Yes”, and the medical support process proceeds to Step ST38.

In Step ST38, the control unit 70C displays a mark corresponding to the importance 104 to be superimposed on the region corresponding to the part for which the omission of recognition has occurred with reference to the importance table 84. For example, in a case in which the first medical support image 41A is displayed on the screen 37, the control unit 70C displays the importance mark 110 corresponding to the importance 104 to be superimposed on a region 109 corresponding to the part for which the omission of recognition has occurred among the plurality of regions 109 in the first medical support image 41A. In addition, in a case in which the second medical support image 41B is displayed on the screen 37, the control unit 70C displays the importance mark 112 corresponding to the importance 104 to be superimposed on a circular mark 116 corresponding to the part for which the omission of recognition has occurred among the plurality of circular marks 116 in the second medical support image 41B. Further, in a case in which the third medical support image 41C is displayed on the screen 37, the control unit 70C displays the importance mark 120 corresponding to the importance 104 to be superimposed on a region 122 corresponding to the part for which the omission of recognition has occurred among the plurality of regions 112 in the second medical support image 41B. After the process in Step ST38 is performed, the medical support process proceeds to Step ST40.

In Step ST40, the control unit 70C ends the image recognition process using the endoscope recognition unit 70B and the part recognition unit 70D. After the process in Step ST40 is performed, the medical support process proceeds to Step ST42.

In Step ST42, the control unit 70C determines whether or not a medical support process end condition is satisfied. An example of the medical support process end condition is a condition that an instruction for the endoscope system 10 to end the medical support process is given (for example, a condition that the receiving device 62 receives an instruction to end the medical support process).

In a case in which the medical support process end condition is not satisfied in Step ST42, the determination result is “No”, and the medical support process proceeds to Step ST10 illustrated in FIG. 13A. In a case in which the medical support process end condition is satisfied in Step ST42, the determination result is “Yes”, and the medical support process ends.

As described above, in the endoscope system 10, the time-series image group 89 is obtained by imaging the inside of the stomach with the camera 48. In addition, the AI-type image recognition process is performed on the time-series image group 89 to acquire the endoscope-related information 90. The endoscopic image 40 is displayed on the screen 36 of the display device 13, and the medical support image 41 is displayed on the screen 37 of the display device 13. The medical support image 41 is referred to by the doctor 14 to check a plurality of parts that are scheduled to be observed during endoscopy.

However, for example, in a case in which the doctor 14 is concentrating on the operation of the endoscope 12, it is difficult to understand all of the content of the medical support image 41. In particular, in a case in which the difficulty of the technique using the endoscope 12 is high or the difficulty of the mental rotation is high, it is difficult to spend a sufficient amount of time to check the medical support image 41.

Therefore, in the endoscope system 10, the first medical support image 41A, the second medical support image 41B, and the third medical support image 41C which have different amounts of visual information are selected according to the endoscope-related information 90 and are displayed on the screen 37. The second medical support image 41B has a larger amount of visual information than the third medical support image 41C, and the first medical support image 41A has a larger amount of visual information than the second medical support image 41B. For example, in a case in which the level at which the doctor 14 concentrates on the operation of the endoscope 12 or the like is high, the third medical support image 41C is displayed on the screen 37. In a case in which the level at which the doctor 14 concentrates on the operation of the endoscope 12 or the like is medium, the second medical support image 41B is displayed on the screen 37. In a case in which the level at which the doctor 14 concentrates on the operation of the endoscope 12 or the like is low, the first medical support image 41A is displayed on the screen 37.

This makes it possible for the doctor 14 to easily understand a plurality of parts in the observation target 21 through the medical support image 41. In addition, the doctor 14 can selectively observe the first medical support image 41A, the second medical support image 41B, and the third medical support image 41C which have different amounts of visual information depending on the situation in which the doctor 14 is placed. That is, as the medical support image 41 to be observed by the doctor 14, a simple medical support image 41 and a detailed medical support image 41 can be used properly depending on the situation in which the doctor 14 is placed.

In addition, in the endoscope system 10, the endoscope-related information 90 includes, for example, the treatment tool information 90A, the operation speed information 90B, the positional information 90C, the shape information 90D, and the fluid delivery information 90E which are information that can specify the difficulty of the technique using the endoscope 12 and/or the difficulty of the mental rotation. Among the first medical support image 41A, the second medical support image 41B, and the third medical support image 41C, the medical support image 41 selected according to, for example, the treatment tool information 90A, the operation speed information 90B, the positional information 90C, the shape information 90D, and the fluid delivery information 90E is displayed on the screen 37. Therefore, the doctor 14 can observe the medical support image 41 with an appropriate amount of information which is matched with the difficulty of the technique using the endoscope 12 and/or the difficulty of the mental rotation among the first medical support image 41A, the second medical support image 41B, and the third medical support image 41C. That is, as the medical support image 41 to be observed by the doctor 14, the simple medical support image 41 and the detailed medical support image 41 can be used properly according to the difficulty of the technique using the endoscope 12 and/or the difficulty of the mental rotation.

In addition, the treatment tool information 90A, the operation speed information 90B, the positional information 90C, the shape information 90D, the fluid delivery information 90E, and the like included in the endoscope-related information 90 are information that can specify the content of the operation on the endoscope 12. Among the first medical support image 41A, the second medical support image 41B, and the third medical support image 41C, the medical support image 41 selected according to, for example, the treatment tool information 90A, the operation speed information 90B, the positional information 90C, the shape information 90D, and the fluid delivery information 90E is displayed on the screen 37. Therefore, the doctor 14 can observe the observation target 21 through the medical support image 41 suitable for the content of the operation on the endoscope 12 among the first medical support image 41A, the second medical support image 41B, and the third medical support image 41C.

In addition, in the endoscope system 10, a schematic perspective view showing a schematic aspect of the stomach is used as the first medical support image 41A. Further, a schematic view showing a schematic aspect of at least one route for observing the stomach is used as the second medical support image 41B. Furthermore, a schematic view showing an aspect in which the stomach is schematically developed is used as the third medical support image 41C. Therefore, as the medical support image 41 to be observed by the doctor 14, a schematic view corresponding to the situation in which the doctor 14 is placed can be provided to the doctor 14.

Moreover, in the endoscope system 10, the plurality of regions 109 included in the first medical support image 41A are classified into the major category and the minor category. In addition, the plurality of circular marks 116 included in the second medical support image 41B are also classified into the major category and the minor category. Further, the plurality of regions 122 included in the third medical support image 41C are also classified into the major category and the minor category. Therefore, the doctor 14 can understand which part of the observation target 21 is classified into the major category and which part of the observation target 21 is classified into the minor category through the medical support image 41 displayed on the screen 37.

Furthermore, in the endoscope system 10, the endoscope recognition unit 70B generates the endoscope-related information 90 on the basis of the time-series image group 89. That is, it is not necessary to input the endoscope-related information 90 from the outside of the endoscope 12 to the endoscope 12. Therefore, it is possible to display the medical support image 41 corresponding to the endoscope-related information 90 on the screen 37 while reducing the time and effort corresponding to at least the input of the endoscope-related information 90 from the outside of the endoscope 12 to the endoscope 12.

In addition, in the endoscope system 10, in the first medical support image 41A, the first observed region and the first unobserved region are displayed to be distinguishable from each other. Further, in the second medical support image 41B, the second observed region and the second unobserved region are displayed to be distinguishable from each other. Furthermore, in the third medical support image 41C, the third observed region and the third unobserved region are displayed to be distinguishable from each other. Therefore, in a case in which the first medical support image 41A is displayed on the screen 37, the doctor 14 can easily understand the first observed region and the first unobserved region. In addition, in a case in which the second medical support image 41B is displayed on the screen 37, the doctor 14 can easily understand the second observed region and the second unobserved region. Further, in a case in which the third medical support image 41C is displayed on the screen 37, the doctor 14 can easily understand the third observed region and the third unobserved region.

In addition, in the endoscope system 10, the first medical support image 41A is displayed as the default medical support image 41 on the screen 37. Then, the first medical support image 41A, the second medical support image 41B, and the third medical support image 41C are selectively displayed on the screen 37, using the first medical support image 41A as a starting point. Therefore, the doctor 14 can perform endoscopy while mainly referring to the first medical support image 41A having the smallest amount of visual information among the first medical support image 41A, the second medical support image 41B, and the third medical support image 41C.

In addition, in the above-described embodiment, an example of the form in which the screens 36 and 37 are displayed on the display device 13 to be comparable has been described. However, this is only an example, and the screen 36 and the screen 37 may be selectively displayed. Further, the size ratio of the screen 36 to the screen 37 may be changed according to, for example, the instruction received by the receiving device 62 and/or the current state of the endoscope 12 (for example, the operation state of the endoscope 12).

In the above-described embodiment, an example of the form in which the part recognition unit 70D performs the AI-type image recognition process has been described. However, the technology of the present disclosure is not limited thereto. For example, the processor 70 may perform an image recognition process of a non-AI type (for example, a template matching type) to recognize the part. In addition, the processor 70 may recognize the part using both the AI-type image recognition process and the non-AI-type image recognition process. Further, it goes without saying that the same is applied to the image recognition process performed by the endoscope recognition unit 70B.

In the above-described embodiment, an example of the form in which the part recognition unit 70D performs the image recognition process on the time-series image group 89 to recognize a part has been described. However, this is only an example, and the image recognition process may be performed on the endoscopic image 40 of a single frame to recognize a part. Further, it goes without saying that the same is applied to the image recognition process performed by the endoscope recognition unit 70B.

In the above-described embodiment, the display aspect of the first importance mark 110A, the display aspect of the second importance mark 110B, and the display aspect of the third importance mark 110C are different depending on the importance 104. However, the technology of the present disclosure is not limited thereto. For example, the display aspect of the first importance mark 110A, the display aspect of the second importance mark 110B, and the display aspect of the third importance mark 110C may be different depending on the type of the unrecognized part. In addition, even in a case in which the display aspect of the importance mark 110 is different depending on the type of the unrecognized part, the display aspect of the importance mark 110 corresponding to the importance 104 may be maintained as in the above-described embodiment. Further, the importance 104 may be changed depending the type of the unrecognized part, and the first importance mark 110A, the second importance mark 110B, and the third importance mark 110C may be selectively displayed according to the changed importance 104. In addition, it goes without saying that the same is applied to the importance marks 112 and 120.

In the above-described embodiment, an example of the form in which the importance 104 is defined at any one of three levels of “high”, “medium”, and “low” levels has been described. However, this is only an example, and the importance 104 may be at one or two of the “high”, “medium”, and “low” levels. In this case, the importance mark 110 may also be determined to be distinguishable for each level of the importance 104. For example, in a case in which the importance 104 is only at the “high” or “medium” level, the first importance mark 110A and the second importance mark 110B may be selectively displayed in the first medical support image 41A according to the importance 104. In addition, the third importance mark 110C may not be displayed in the first medical support image 41A. In addition, the importance 104 may be divided into four or higher levels. In this case, the importance mark 110 may also be determined to be distinguishable for each level of the importance 104. In addition, it goes without saying that the same is applied to the importance marks 112 and 120.

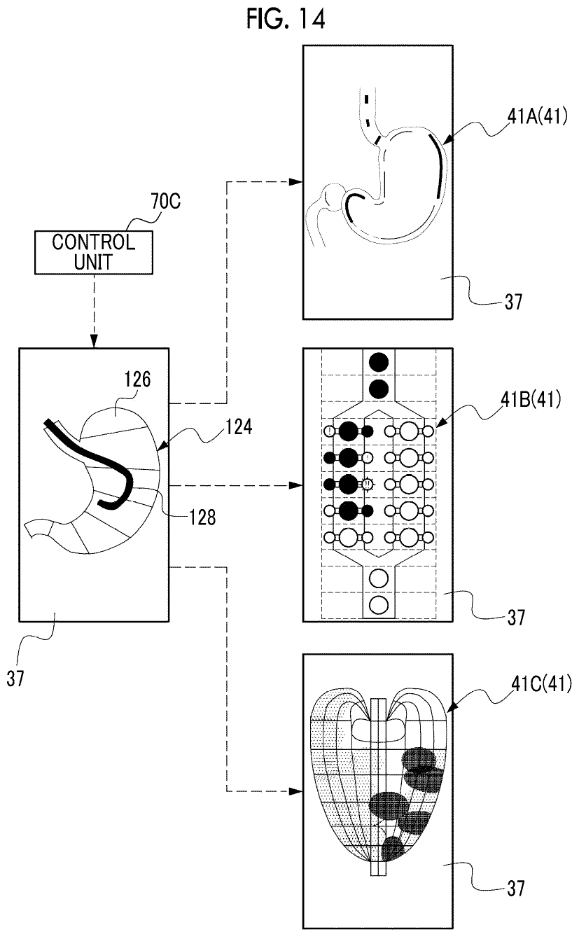

In the above-described embodiment, an example of the form in which the first medical support image 41A, the second medical support image 41B, and the third medical support image 41C are selectively displayed on the screen 37 using the first medical support image 41A as a starting point has been described. However, the technology of the present disclosure is not limited thereto. For example, as illustrated in FIG. 14, a reference image 124, the first medical support image 41A, the second medical support image 41B, and the third medical support image 41C may be selectively displayed on the screen 37 using the reference image 124 as a starting point. The reference image 124 is an example of a “first image” according to the technology of the present disclosure. The reference image 124 is an image including a plurality of regions 126 that correspond to a plurality of parts in the observation target 21 and an insertion portion image 128. The reference image 124 is divided into the plurality of regions 126. In the reference image 124, the plurality of regions 126 and the insertion portion image 128 are represented to be comparable. The insertion portion image 128 is an image that imitates the insertion portion 44. The shape and position of the insertion portion image 128 are linked to the actual shape and position of the insertion portion 44.

The actual shape and position of the insertion portion 44 are specified by performing the AI-type image recognition process. For example, the control unit 70C specifies the actual shape and position of the insertion portion 44 by performing the process using the trained model on the content of the operation of the insertion portion 44 and the endoscopic images 40 of one or more frames, generates the insertion portion image 128 on the basis of the specification result, and displays the insertion portion image 128 to be superimposed on the reference image 124 on the screen 37.

Here, for example, the trained model used by the control unit 70C is obtained by performing machine learning on the neural network using training data in which the content of the operation of the insertion portion 44, images corresponding to the endoscopic images 40 of one or more frames, and the like are example data and the shape and position of the insertion portion 44 are correct answer data.

In the example illustrated in FIG. 14, an example of the form in which the reference image 124, the first medical support image 41A, the second medical support image 41B, and the third medical support image 41C are selectively displayed on the screen 37 has been described. However, the reference image 124 and the first medical support image 41A, the second medical support image 41B, or the third medical support image 41C may be displayed in a state in which they are arranged side by side (that is, in a state in which they are comparable). In this case, for example, as the medical support image 41 displayed side by side with the reference image 124, using the reference image 124 and the first medical support image 41A as the starting point, the first medical support image 41A, the second medical support image 41B, and the third medical support image 41C may be selectively displayed.

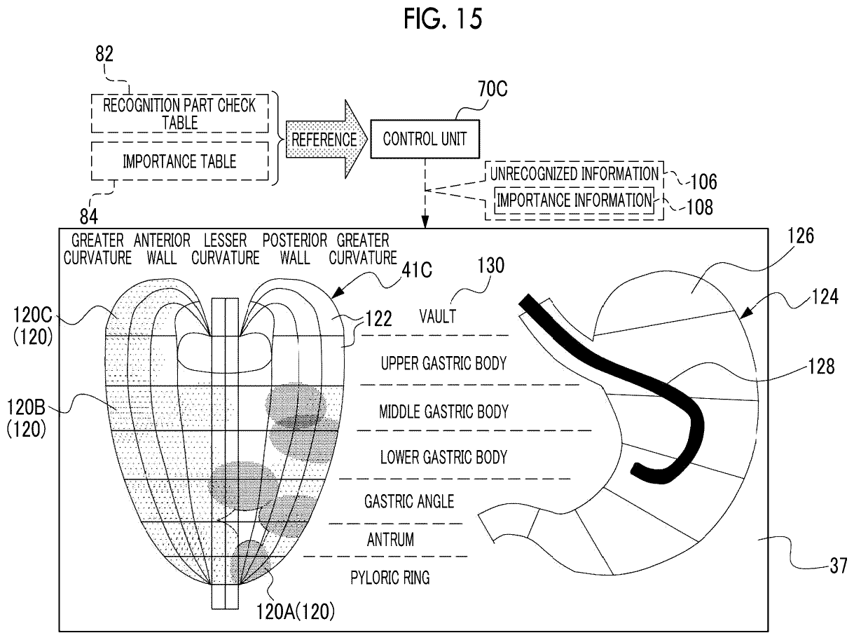

In a case in which the reference image 124 and the third medical support image 41C are displayed side by side on the screen 37, for example, information 130 (text in an example illustrated in FIG. 15) that can specify the positions of the vault, the upper gastric body, the middle gastric body, the lower gastric body, the gastric angle, the antrum, and the pyloric ring may be displayed on the screen 37 as illustrated in FIG. 15. In this case, the information 130 may be displayed such that the plurality of regions 126 included in the reference image 124 are associated with the plurality of regions 122 included in the third medical support image 41C.

The reference image 124 and at least one medical support image 41 may be selected according to the endoscope-related information 90 in the same manner as described in the above-described embodiment, and the selected images may be displayed on the screen 37. Therefore, the doctor 14 can understand a plurality of parts in the observation target 21 and can understand the position of the endoscope 12 (here, for example, the insertion portion 44) in the observation target 21.

In the above-described embodiment, an example of the form in which the endoscope-related information 90 includes the treatment tool information 90A, the operation speed information 90B, the positional information 90C, the shape information 90D, and the fluid delivery information 90E. However, the technology of the present disclosure is not limited thereto. For example, the endoscope-related information 90 may include operator information that can identify the operator of the endoscope 12. An example of the operator information is an identifier that can identify each doctor 14 or information indicating whether or not the operator has a predetermined level of skill in the operation of the endoscope 12. Since the operator information is included in the endoscope-related information 90, the medical support image 41 corresponding to the operator information is selected from the first medical support image 41A, the second medical support image 41B, and the third medical support image 41C, and the selected medical support image 41 is displayed on the screen 37. The medical support image 41 displayed on the screen 37 is an image suitable for the operator.

Therefore, for example, in a case in which the doctor 14 who is not used to operating the endoscope 12 operates the endoscope 12, the medical support image 41 (for example, the second medical support image 41B or the third medical support image 41C) having a large amount of information is displayed on the screen 37. In addition, for example, in a case in which the doctor 14 who is used to operating the endoscope 12 operates the endoscope 12, the medical support image 41 (for example, the first medical support image 41C) having a small amount of information is displayed on the screen 37. As described above, the inclusion of the operator information in the endoscope-related information 90 makes it possible to provide the medical support image 41 including the amount of information suitable for the doctor 14 to the doctor 14.

In the above-described embodiment, an example of the form in which the difficulty 92 is calculated on the basis of the information included in the endoscope-related information 90 has been described. However, the technology of the present disclosure is not limited thereto. For example, the difficulty 92 may be calculated from the arithmetic expression 93 on the basis of the information included in the endoscope-related information 90 and the part information 94. In this case, for example, the high difficulty 92A may be calculated for the part information 94 related to a part that is difficult to observe (for example, a part extending across a joint portion of the esophagus and the stomach), or the low difficulty 92C may be calculated for the part information 94 related to a part that is easy to observe.

In the above-described embodiment, an example of the form in which the importance 104 assigned to a plurality of parts is determined according to the data of the past examination performed on the plurality of parts has been described. However, the technology of the present disclosure is not limited thereto. For example, the importance 104 assigned to the plurality of parts may be determined according to the position of the unrecognized part in the stomach. The omission of the recognition of a part that is spatially farthest from the position of the distal end part 46 by the part recognition unit 70D is more likely to occur than the omission of the recognition of a part that is spatially closer to the position of the distal end part 46. Therefore, an example of the position of the unrecognized part in the stomach is the position of the unrecognized part that is spatially farthest from the position of the distal end part 46. In this case, the position of the unrecognized part that is spatially farthest from the position of the distal end part 46 changes depending on the position of the distal end part 46. Therefore, the importance 104 assigned to a plurality of parts changes depending on the position of the distal end part 46 and the position of the unrecognized part in the stomach. As described above, since the importance 104 assigned to the plurality of parts is determined according to the position of the unrecognized part in the stomach, it is possible to suppress the omission of the recognition of the part with the high importance 104 determined according to the position of the unrecognized part in the stomach by the part recognition unit 70D.

In the above-described embodiment, an example of the form in which the importance 104 assigned to the plurality of parts is determined in response to an instruction given from the outside has been described. However, the technology of the present disclosure is not limited thereto. For example, the importance 104 corresponding to a part that is scheduled to be recognized by the part recognition unit 70D before a designated part (for example, a part corresponding to a predetermined checkpoint) among a plurality of parts may be set to be higher than the importance 104 corresponding to a part that is scheduled to be recognized after the designated part among the plurality of parts. This makes it possible to suppress the omission of the recognition of the part that is scheduled to be recognized by the part recognition unit 70D before the designated part.

In the above-described embodiment, an example of the form in which the unrecognized part is set regardless of the part classified into the major category and the part classified into the minor category among a plurality of parts has been described. However, the technology of the present disclosure is not limited thereto. For example, the omission of the recognition of the part classified into the minor category by the part recognition unit 70D is more likely to occur than the omission of the recognition of the part classified into the major category by the part recognition unit 70D. Therefore, the unrecognized part may be set only for the part classified into the minor category among the plurality of parts. In this case, the omission of the recognition by the part recognition unit 70D can be less likely to occur as compared to a case in which the omission of the recognition of both the part classified into the major category and the part classified into the minor category by the part recognition unit 70D is suppressed.

In the above-described embodiment, an example of the form has been described in which, in a case in which the part classified into the minor category has not been recognized by the part recognition unit 70D, the unrecognized information 106 is output on condition that the part recognition unit 70D recognizes a part classified into the major category scheduled to be recognized by the part recognition unit 70D after the part which has not been recognized by the part recognition unit 70D. However, the technology of the present disclosure is not limited thereto.

For example, in a case in which the part classified into the minor category has not been recognized by the part recognition unit 70D, the unrecognized information 106 may be output on condition that the part recognition unit 70D recognizes a part classified into the minor category scheduled to be recognized by the part recognition unit 70D after the part which has not been recognized by the part recognition unit 70D (that is, the part classified into the minor category). In this case, in a scene in which there is a high possibility that the omission of recognition will occur for a part (here, for example, a part classified into the minor category) in the observation target 21, the doctor 14 can understand that the omission of recognition has occurred for the part in the observation target 21.

In addition, for example, in a case in which the part classified into the minor category has not been recognized by the part recognition unit 70D, the unrecognized information 106 may be output on condition that the part recognition unit 70D recognizes a plurality of parts classified into the minor category scheduled to be recognized by the part recognition unit 70D after the part which has not been recognized by the part recognition unit 70D (that is, the part classified into the minor category). In this case, in a scene in which there is a high possibility that the omission of the recognition of a part (here, for example, a part classified into the minor category) in the observation target 21 will occur, the doctor 14 can understand that the omission of the recognition of the part in the observation target 21 has occurred.

In the above-described embodiment, an example of the form in which the unrecognized information 106 is output from the control unit 70C to the display device 13 has been described. However, the technology of the present disclosure is not limited thereto. For example, the unrecognized information 106 may be stored in headers or the like of various images such as the endoscopic images 40. For example, in a case in which the part which has not been recognized by the part recognition unit 70D is classified into the minor category, the fact that the part is classified into the minor category and/or information that can specify the part may be stored in the headers or the like of various images such as the endoscopic images 40. In addition, for example, in a case in which the part which has not been recognized by the part recognition unit 70D is classified into the major category, the fact that the part is classified into the major category and/or information that can specify the part may be stored in the headers or the like of various images such as the endoscopic images 40.

Further, a recognition order including the major category and the minor category (that is, the order of the parts recognized by the part recognition unit 70D) and/or information related to a finally unrecognized part (that is, the part which has not been recognized by the part recognition unit 70D) may be transmitted to an examination system that is connected to the endoscope 12 such that it can communicate therewith and may be stored as examination data by the examination system or may be posted in an examination diagnosis report. In addition, information indicating the observation results of a checkpoint among a plurality of parts (for example, the unrecognized information 106 or information based on the unrecognized information 106) and information indicating comprehensive observation results (the observation results of the part classified into the major category and/or the part classified into the minor category) may be stored in association with examination data (for example, images obtained by performing the examination and/or information related to the examination).

Further, information indicating an observation order (that is, an observation route) (for example, information related to the order of the parts recognized by the part recognition unit 70D) may be stored in association with the examination data. Furthermore, in addition to an examination ID, information of, for example, an observation part (for example, a part recognized by the part recognition unit 70D) may be recorded on the headers or the like of various images such as the endoscopic images 40.

In addition, in the next examination, the previous observation route or the like and/or a comprehensive map (for example, the first medical support image 41A, the second medical support image 41B, and/or the third medical support image 41C) may be displayed on the display device 13 or the like.

In the above-described embodiment, an example of the form has been described in which the camera 48 sequentially images a plurality of parts on the greater-curvature-side route 114A from the upstream side (that is, the entrance side of the stomach) to the downstream side of the stomach (that is, the exit side of the stomach) and sequentially images the lesser-curvature-side route 114B from the upstream side to the downstream side of the stomach (that is, the parts are imaged along the scheduled recognition order 102). However, the technology of the present disclosure is not limited thereto. For example, in a case in which the part recognition unit 70D sequentially recognizes a first part (for example, the posterior wall of the upper gastric body) on the upstream side in an insertion direction of the insertion portion 44 inserted into the stomach and a second part (for example, the posterior wall of the lower gastric body) on the downstream side, the processor 70 estimates that imaging is performed along the first route (here, for example, the greater-curvature-side route 114A) determined from the upstream side to the downstream side of the insertion portion 44, and the unrecognized information 106 is output along the first route. In addition, for example, in a case in which the part recognition unit 70D sequentially recognizes a third part (for example, the posterior wall of the lower gastric body) on the downstream side in the insertion direction of the insertion portion 44 inserted into the stomach and a fourth part (for example, the posterior wall of the upper gastric body) on the upstream side, the processor 70 estimates that imaging is performed along the second route (here, for example, the lesser-curvature-side route 114B) determined from the downstream side to the upstream side of the insertion portion 44, and the unrecognized information 106 is output along the second route. Therefore, it is possible to easily specify whether the part on the greater-curvature-side route 114A is not recognized by the part recognition unit 70D or the part on the lesser-curvature-side route 114B is not recognized by the part recognition unit 70D.

In addition, here, the greater-curvature-side route 114A is given as an example of the first route, and the lesser-curvature-side route 114B is given as an example of the second route. However, the first route may be the lesser-curvature-side route 114B, and the first route may be the greater-curvature-side route 114A. Further, here, the upstream side in the insertion direction indicates the entrance side of the stomach (that is, the esophageal side), and the downstream side in the insertion direction indicates the exit side of the stomach (that is, the duodenal side).

In the above-described embodiment, an example of the form in which the endoscope-related information 90 is obtained from the first trained model 78 has been described. However, the technology of the present disclosure is not limited thereto. For example, the endoscope-related information 90 may be input to the control device 22 through the receiving device 62 or may be input to the control device 22 through an external device (for example, a tablet terminal, a personal computer, or a server) that is connected to the control device 22 such that it can communicate therewith.

In the above-described embodiment, an example of the form in which the medical support process is performed by the processor 70 of the computer 64 included in the endoscope 12 has been described. However, the technology of the present disclosure is not limited thereto. The device that performs the medical support process may be provided outside the endoscope 12. An example of the device provided outside the endoscope 12 is at least one server and/or at least one personal computer that is connected to the endoscope 12 such that it can communicate therewith. In addition, the medical support process may be dispersively performed by a plurality of devices.

Further, in the above-described embodiment, an example of the form in which the medical support processing program 76 is stored in the NVM 74 has been described. However, the technology of the present disclosure is not limited thereto. For example, the medical support processing program 76 may be stored in a portable non-transitory storage medium such as an SSD or a USB memory. The medical support processing program 76 stored in the non-transitory storage medium is installed in the computer 64 of the endoscope 12. The processor 70 performs the medical support process according to the medical support processing program 76.

In addition, the medical support processing program 76 may be stored in a storage device of another computer or a server that is connected to the endoscope 12 through a network. Then, the medical support processing program 76 may be downloaded and installed in the computer 64 in response to a request from the endoscope 12.

In addition, all of the medical support processing program 76 does not need to be stored in the storage device of another computer or the server connected to the endoscope 12 or the NVM 74, and a portion of the medical support processing program 76 may be stored therein.

The following various processors can be used as hardware resources for performing the medical support process. An example of the processor is a CPU which is a general-purpose processor that executes software, that is, a program, to function as the hardware resource performing the medical support process. In addition, an example of the processor is a dedicated electronic circuit which is a processor having a dedicated circuit configuration designed to perform a specific process, such as an FPGA, a PLD, or an ASIC. Any processor has a memory provided therein or connected thereto. Any processor uses the memory to perform the medical support process.

The hardware resource for performing the medical support process may be configured by one of the various processors or by a combination of two or more processors of the same type or different types (for example, a combination of a plurality of FPGAs or a combination of a CPU and an FPGA). Further, the hardware resource for performing the medical support process may be one processor.

A first example of the configuration in which the hardware resource is configured by one processor is an aspect in which one processor is configured by a combination of one or more CPUs and software and functions as the hardware resource for performing the medical support process. A second example of the configuration is an aspect in which a processor that implements the functions of the entire system including a plurality of hardware resources for performing the medical support process using one IC chip is used. A representative example of this aspect is an SoC. As described above, the medical support process is achieved using one or more of the various processors as the hardware resource.

In addition, specifically, an electronic circuit obtained by combining circuit elements, such as semiconductor elements, can be used as the hardware structure of the various processors. Further, the above-described medical support process is only an example. Therefore, it goes without saying that unnecessary steps may be deleted, new steps may be added, or the processing order may be changed, without departing from the gist.

The content described and illustrated above is a detailed description of portions related to the technology of the present disclosure and is only an example of the technology of the present disclosure. For example, the description of the configurations, functions, operations, and effects is the description of examples of the configurations, functions, operations, and effects of the portions related to the technology of the present disclosure. Therefore, it goes without saying that unnecessary portions may be deleted or new elements may be added or replaced in the content described and illustrated above, without departing from the gist of the technology of the present disclosure. In addition, the description of, for example, common technical knowledge that does not need to be particularly described to enable the implementation of the technology of the present disclosure is omitted in the content described and illustrated above in order to avoid confusion and to facilitate the understanding of the portions related to the technology of the present disclosure.

In the specification, “A and/or B” is synonymous with “at least one of A or B”. That is, “A and/or B” means only A, only B, or a combination of A and B. Further, in the specification, the same concept as “A and/or B” is applied to a case in which the connection of three or more matters is expressed by “and/or”.

All of the documents, the patent applications, and the technical standards described in the specification are incorporated by reference herein to the same extent as each individual document, each patent application, and each technical standard is specifically and individually stated to be incorporated by reference.

What is claimed is:

1. A medical support device comprising:
a processor,
wherein the processor acquires endoscope-related information that is related to an endoscope and displays, on a display device, at least one image selected according to the endoscope-related information among a plurality of images in which an observation target observed through the endoscope is divided into a plurality of regions and which are represented in different aspects.

2. The medical support device according to claim 1,
wherein the plurality of images have different amounts of visual information.

3. The medical support device according to claim 2,
wherein the amount of information is classified into a first amount of information and a second amount of information that is less than the first amount of information,
the endoscope-related information includes difficulty information that is capable of specifying a difficulty of a technique using the endoscope and/or a difficulty of mental rotation, and
the processor switches between the image with the first amount of information and the image with the second amount of information as the image to be displayed on the display device, according to the difficulty information.

4. The medical support device according to claim 1,
wherein the plurality of images are classified into a simple image in a simple format and a detailed image in a format that is more detailed than the simple image.

5. The medical support device according to claim 4,
wherein the endoscope-related information includes difficulty information that is capable of specifying a difficulty of a technique using the endoscope and/or a difficulty of mental rotation, and
the processor switches between the simple image and the detailed image as the image to be displayed on the display device, according to the difficulty information.

6. The medical support device according to claim 1,
wherein the observation target is a luminal organ,
the plurality of images are a plurality of schematic views including a first schematic view, a second schematic view, and a third schematic view,
the first schematic view is a view showing a schematic aspect of at least one route for observing the luminal organ,
the second schematic view is a perspective view showing a schematic aspect of the luminal organ, and
the third schematic view is a view showing an aspect in which the luminal organ is schematically developed.

7. The medical support device according to claim 6,
wherein the plurality of regions are classified into a major category and a minor category included in the major category, and
in at least one of the first schematic view, the second schematic view, or the third schematic view, the major category, the minor category, or both the major category and the minor category are represented.

8. The medical support device according to claim 1,
wherein the endoscope-related information includes information that is capable of specifying content of an operation corresponding to the endoscope.

9. The medical support device according to claim 1,
wherein the endoscope-related information includes information that is capable of specifying an operator of the endoscope.

10. The medical support device according to claim 1,
wherein the endoscope generates an endoscopic image including the observation target, and
the endoscope-related information is information generated on the basis of the endoscopic image.

11. The medical support device according to claim 1,
wherein the endoscope generates an endoscopic image including the observation target,
the processor classifies the plurality of regions into an observed region which has been observed through the endoscope and an unobserved region which has not been observed through the endoscope on the basis of the endoscopic image, and
the observed region and the unobserved region are displayed to be distinguishable from each other in the at least one image.

12. The medical support device according to claim 11,
wherein the observation target is a luminal organ, and
the plurality of images include a first image in which a position of the endoscope in the luminal organ and the plurality of regions are comparable from each other and a second image in which the observed region and the unobserved region in the luminal organ are distinguishable from each other.

13. The medical support device according to claim 11,
wherein the observation target is a luminal organ, and
the plurality of images include a third image in which the observed region and the unobserved region in the luminal organ are distinguishable from each other and at least one fourth image in which the observed region and the unobserved region in the luminal organ are distinguishable from each other in more detail than the third image.

14. The medical support device according to claim 13,
wherein the plurality of images include, as the fourth image, a fourth schematic view showing a schematic aspect of at least one route for observing the luminal organ and a fifth schematic view showing an aspect in which the luminal organ is schematically developed.

15. The medical support device according to claim 13,
wherein the third image and the at least one fourth image are selectively displayed on the display device, using the third image as a starting point.

16. The medical support device according to claim 1,
wherein the processor outputs unobserved information capable of specifying that an unobserved region, which has not been observed through the endoscope, is present in the plurality of regions along a first route determined from an upstream side to a downstream side in an insertion direction of the endoscope inserted into a body in a case in which a first part on the upstream side and a second part on the downstream side in the insertion direction are sequentially recognized, and outputs the unobserved information along a second route determined from the downstream side to the upstream side in the insertion direction in a case in which a third part on the downstream side and a fourth part on the upstream side in the insertion direction are sequentially recognized.

17. An endoscope comprising:
the medical support device according to claim 1; and
an image acquisition device that acquires an endoscopic image including the observation target.

18. A medical support method comprising:
acquiring endoscope-related information that is related to an endoscope; and
displaying, on a display device, at least one image selected according to the endoscope-related information among a plurality of images in which an observation target observed through the endoscope is divided into a plurality of regions and which are represented in different aspects.

19. A non-transitory computer-readable storage medium storing a program executable by a computer to perform a process comprising:
acquiring endoscope-related information that is related to an endoscope; and
displaying, on a display device, at least one image selected according to the endoscope-related information among a plurality of images in which an observation target observed through the endoscope is divided into a plurality of regions and which are represented in different aspects.
